(19) United States
(12) Patent Application Publication
(10) Pub. No.: US 2023/0190728 A1
(43) Pub. Date: Jun. 22, 2023

(54) NEW THERAPEUTIC USES OF COMPOUNDS

(71) Applicant: PROXIMAGEN, LLC, Plymouth, MN (US)

(72) Inventors: William Pullman, Plymouth, MN (US); Peter Van Ess, Plymouth, MN (US)

(21) Appl. No.: 17/913,379

(22) PCT Filed: Mar. 25, 2021
(86) PCT No.: PCT/US2021/024193

§ 371 Date: Sep. 21, 2022

Related U.S. Application Data
(60) Provisional application No. 63/000,450, filed on Mar. 26, 2020.

(51) Int. Cl. A61K 31/4545; A61P 29/00; A61P 11/00; A61P 31/14; A61K 31/5377; A61K 31/496; A61K 31/551

(52) U.S. Cl. CPC A61K 31/4545; A61K 31/496; A61K 31/551; A61K 31/5377; A61P 11/00; A61P 29/00; A61P 31/14

(57) ABSTRACT

The present invention relates to the treatment and prevention of pulmonary inflammation using a compound of Formula (I) or a pharmaceutically acceptable salt, solvate, hydrate, N-oxide, and/or prodrug thereof. The pulmonary inflammation may be associated with acute lung injury (ALI) and/or acute respiratory distress syndrome (ARDS), which in turn may be associated with viral induced cytokine surge. Such diseases and conditions may be caused by a coronavirus, i.e., severe acute respiratory syndrome coronavirus 2 (SARS-CoV-2), severe acute respiratory syndrome coronavirus (SARS-CoV), or Middle East respiratory syndrome coronavirus (MERS-CoV). This abstract is intended as a scanning tool for purposes of searching in the particular art and is not intended to be limiting of the present invention.

### CROSS-REFERENCE TO RELATED APPLICATIONS

This Application claims the benefit of U.S. Application No. 63/000,450, filed on Mar. 26, 2020, the contents of which are hereby incorporated by reference in their entirety.

### FIELD OF INVENTION

The present invention relates to the treatment and prevention of pulmonary inflammation using a compound of Formula (I) or a pharmaceutically acceptable salt, solvate, hydrate, N-oxide, and/or prodrug thereof. The pulmonary inflammation may cause acute lung injury (ALI) and/or acute respiratory distress syndrome (ARDS). The pulmonary inflammation may be caused by cytokine surge, such as viral induced cytokine surge. Such diseases and conditions may be caused by a coronavirus, i.e. severe acute respiratory syndrome coronavirus 2 (SARS-CoV-2), severe acute respiratory syndrome coronavirus (SARS-CoV), or Middle East respiratory syndrome coronavirus (MERS-CoV).

### BACKGROUND

Since their initial discovery in the 1960s, a variety of human-infecting coronaviruses (hCov) have been characterised. From the family Coronaviridae, these viruses primarily infect the upper respiratory and gastrointestinal tracts to cause respiratory infections. In general, coronaviruses infecting humans can be classified into low pathogenic human coronaviruses, which include HCoV-229E, HCoVOC43, HCoV-NL63, and HCoV-HKU and highly pathogenic hCoVs such as SARS-CoV, MERS-CoV, and the 2019 outbreak strain of SARS-CoV-2 (2019-nCov or COVID-19).

Coronaviruses are enveloped single-stranded RNA viruses named so for their crown-like surface structure composed of spike (S), envelope (E), membrane (M) and nucleocapsid (N) proteins. The spike protein in particular is responsible for the action of entering a host cell, wherein the coronavirus is able to transcribe its RNA for intracytoplasmic replication. Indeed, coronaviruses have a unique ability to replicate and survive in the intracellular space of a macrophage, whereby multiple encoded interferon antagonists are thought to hinder the activation of type I interferon (IFN) and interferon stimulated genes (ISGs), dampening the host immune response and contributing to the resultant pathogenesis of the virus (Rose et al. 2010, Journal of Virology 84 (11): 5656-5669).

Upon genome replication and polyprotein formation, the viruses assemble and are released from the infected cell to further disseminate. Transmission between hosts is considered to occur primarily by contact with respiratory droplets infected with such viral particles, generated through sneezing and coughing.

Coronaviruses can emerge from animal reservoirs to cause significant epidemics in humans, as exemplified by SARS-CoV in 2002-2003 and MERS-CoV, which was recognised as an emerging virus in 2012, each of which resulted in over 8000 infections and 774 deaths, and 2500 infections and 862 deaths, per respective outbreak (WHO, 2020). Declared a global emergency by the World Health Organisation (WHO), the newly discovered and rapidly disseminating SARS-CoV-2, sharing ~70% genetic similarity to the SARS-CoV, is likely to have similar epidemiological characteristics and thus presents a pressing area of healthcare concern. Crucially, there are no vaccines or antiviral drugs suitable for the prevention or treatment of human coronavirus infections at this time (Habibzadeh & Stoneman 2020, Int J Occup Environ Med 11 (2): 65-71).

A SARS-CoV-2 health emergency challenge is evident of a lack of effective treatments or vaccines, which thus leads to a high unmet need for the protection of high-risk populations, including health care workers and patients in acute danger of nosocomial transmission of SARS-CoV-2, or in other confined spaces, such as during quarantine settings.

Low pathogenic hCoVs infect upper airways and cause seasonal mild to moderate cold-like respiratory illnesses in healthy individuals. In contrast, the high pathogenic hCoVs are responsible for acute and chronic diseases of the lower respiratory tract, hepatic, gastrointestinal and neurological systems. These lower respiratory tract infections can cause severe pulmonary inflammation (hyperinflammation) which may manifest itself as pneumonia, pneumonia-type symptoms, or pneumonitis. The pulmonary inflammation can lead to ALI and ARDS, resulting in high morbidity and mortality. ARDS is a life-threatening condition where the lungs cannot provide the body’s vital organs with enough oxygen. ALI and ARDS can be fatal, particularly for those with underlying health conditions.

Although viral factors regulating the pro-inflammatory response of neutrophils and monocyte-macrophages remain to be identified, the E protein of hCoV has been shown to enhance pro-inflammatory cytokine and chemokine and inflammasome activity via its ion channel activity. Higher virus titers and dysregulated cytokine/chemokine responses cause a “cytokine storm” with lung immunopathological changes following hCoV infection (Channappanavar et al., Semin Immunopathol 2017, 39, 529-539).

There is therefore a clear and unmet need for new pharmaceutical compounds that treat pulmonary inflammation, particularly hyperinflammation, such as that which causes ALI and ARDS, i.e., pulmonary inflammation caused by hCoV.

### SUMMARY

The invention is based upon the surprising finding that administration of a semicarbazide sensitive amine oxidase (“SSAO”) inhibitor, such as a compound of Formula (I), can result in effect treatment and prevention of pulmonary inflammation. In view of this, in a first aspect of the invention there is provided a method for treating or preventing pulmonary inflammation in a subject, the method comprising the step of administering to the subject a compound of Formula (I), or a pharmaceutically acceptable salt, solvate, hydrate, N-oxide, and/or prodrug thereof. The compound of Formula (I) is as herein defined below.

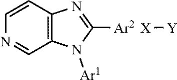

In a second aspect of the invention, there is provided a compound of Formula (I), or a pharmaceutically acceptable salt, solvate, hydrate, N-oxide, and/or prodrug thereof, for use in the treatment or prevention of pulmonary inflammation. The compound of Formula (I) is as herein defined below.

In a third aspect of the invention, there is provided use of a compound of Formula (I), or a pharmaceutically acceptable salt, solvate, hydrate, N-oxide, and/or prodrug thereof, in the manufacture of a medicament for use in the treatment or prevention of pulmonary inflammation. The compound of Formula (I) is as herein defined below.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative data illustrating the effect of oral pre-treatment with Example 7 on LPS-induced pulmonary Neutrophil infiltration. Example 7 decreases neutrophil infiltration in lung tissue indicating a reduction in pulmonary inflammation.

FIG. 2 shows representative data illustrating the effect of oral pre-treatment with Example 7 on LPS-induced pulmonary lavage TNFa content. Example 7 decreases TNFa content in pulmonary lavage indicating a reduction in pulmonary inflammation.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention is based upon the surprising finding that administration of an SSAO inhibitor, such as a compound of Formula (I), can result in effect treatment and prevention of pulmonary inflammation in a subject. It is preferable that the subject is a mammalian subject, and in particular a human. The pulmonary inflammation may cause ALI and/or ARDS. The pulmonary inflammation may be caused by cytokine surge, such as viral induced cytokine surge. In certain cases, the viral-induced cytokine surge may lead to increased inflammatory macrophage-monocyte and neutrophil infiltration. Such diseases and conditions may be caused by a coronavirus, i.e., SARS-CoV-2, SARS-CoV, or MERS-CoV.

Semicarbazide-sensitive amine oxidase (SSAO) activity is an enzyme activity expressed by Vascular Adhesion Protein-1 (VAP-1) or Amine Oxidase, Copper Containing 3 (AOC3), belongs to the copper-containing amine oxidase family of enzymes (EC. 1.4.3.6). Inhibitors of the SSAO enzyme may also modulate the biological functions of the VAP-1 protein. Members of this enzyme family are sensitive to inhibition by semicarbazide and utilize cupric ion and protein-derived topa quinone (TPQ) cofactor in the oxidative deamination of primary amines to aldehydes, hydrogen peroxide, and ammonia according to the following reaction:

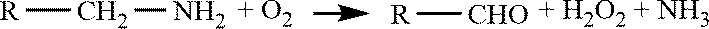

Known substrates for human SSAO include endogenous methylamine and aminoacetone as well as some xenobiotic amines such as benzylamine (Lyles, Int. J. Biochem. Cell Biol. 1996, 28, 259-274; Klinman, Biochim. Biophys. Acta 2003, 1647(1-2), 131-137; Matyus et al., Curr. Med. Chem. 2004, 11(10), 1285-1298; O’Sullivan et al., Neurotoxicology 2004, 25(1-2), 303-315). In analogy with other copper-containing amine oxidases, DNA-sequence analysis and structure determination suggest that the tissue-bound human SSAO is a homodimeric glycoprotein consisting of two 90-100 kDa subunits anchored to the plasma membrane by a single N-terminal membrane spanning domain (Morris et al., J. Biol. Chem. 1997, 272, 9388-9392; Smith et al., J. Exp. Med. 1998, 188, 17-27; Airenne et al., Protein Science 2005, 14, 1964-1974; Jakobsson et al., Acta Crystallogr. D Biol. Crystallogr. 2005, 61(Pt 11), 1550-1562).

SSAO activity has been found in a variety of tissues including vascular and non-vascular smooth muscle tissue, endothelium, and adipose tissue (Lewinsohn, Braz. J. Med. Biol. Res. 1984, 17, 223-256; Nakos & Gossrau, Folia Histochem. Cytobiol. 1994, 32, 3-10; Yu et al., Biochem. Pharmacol. 1994, 47, 1055-1059; Castillo et al., Neurochem. Int. 1998, 33, 415-423; Lyles & Pino, J. Neural. Transm. Suppl. 1998, 52, 239-250; Jaakkola et al., Am. J. Pathol. 1999, 155, 1953-1965; Morin et al., J. Pharmacol. Exp. Ther. 2001, 297, 563-572; Salmi & Jalkanen, Trends Immunol. 2001, 22, 211-216). In addition, SSAO protein is found in blood plasma and this soluble form appears to have similar properties as the tissue-bound form (Yu et al., Biochem. Pharmacol. 1994, 47, 1055-1059; Kurkijärvi et al., J. Immunol. 1998, 161, 1549-1557). It has been shown that circulating human and rodent SSAO originates from the tissue-bound form (Göktürk et al., Am. J. Pathol. 2003, 163(5), 1921-1928; Abella et al., Diabetologia 2004, 47(3), 429-438; Stolen et al., Circ. Res. 2004, 95(1), 50-57), whereas in other mammals the plasma/serum SSAO is also encoded by a separate gene called AOC4 (Schwelberger, J. Neural. Transm. 2007, 114(6), 757-762).

The precise physiological role of this abundant enzyme has yet to be fully determined, but it appears that SSAO and its reaction products may have several functions in cell signaling and regulation. For example, findings suggest that SSAO plays a role in both GLUT4-mediated glucose uptake (Enrique-Tarancon et al., J. Biol. Chem. 1998, 273, 8025-8032; Morin et al., J. Pharmacol. Exp. Ther. 2001, 297, 563-572) and adipocyte differentiation (Fontana et al., Biochem. J. 2001, 356, 769-777; Mercier et al., Biochem. J. 2001, 358, 335-342). A link between SSAO and angiogenesis has been discovered (Noda et al., FASEB J. 2008, 22(8), 2928-2935), and based on this link it is expected that inhibitors of SSAO have an anti-angiogenic effect.

WO 2007/146188 teaches that blocking SSAO activity inhibits leucocyte recruitment, reduces the inflammatory response, and is expected to be beneficial in prevention and treatment of seizures, for example, in epilepsy.

O’Rourke et al (J. Neural. Transm. 2007, 114(6), 845-9) examined the potential of SSAO inhibitors in neurological diseases, having previously demonstrated the efficacy of SSAO inhibition in a rat model of stroke. An SSAO inhibitor is tested on relapsing-remitting experimental autoimmune encephalomyelitis (EAE), a mouse model that shares many characteristics with human multiple sclerosis. The data demonstrates the potential clinical benefit of small molecule anti-SSAO therapy in this model and therefore in treatment of human multiple sclerosis.

Small molecules of different structural classes have previously been disclosed as SSAO inhibitors, for example in WO 02/38153 (tetrahydroimidazo[4,5-c]pyridine derivatives), in WO 03/006003 (2-indanylhydrazine derivatives), in WO 2005/014530 (allylhydrazine and hydroxylamine (aminooxy) compounds) and in WO 2007/120528 (allylamino compounds). Additional SSAO inhibitors are disclosed in PCT/EP2009/062011 and PCT/EP2009/062018. Additional SSAO inhibitors are disclosed in PCT/GB2012/052265.

WO 2013/078254 discloses compounds apparently useful as inhibitors of serine/threonine protein kinases. The compounds are structurally related to the claimed compounds, and have a bicyclic heteroaryl ring system substituted with a phenyl-cyclobutaneamine substituent.

WO 2014/140592 relates to SSAO inhibitors with biological, pharmacological, and pharmacokinetic characteristics that make them suitable for use as prophylactic or therapeutic agents in a wide range of human inflammatory diseases and immune disorders, such as multiple sclerosis, arthritis and vasculitis.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, a “subject” may be a subject in need thereof, in which case the method of the invention is for the treatment of a disease or condition. In the case of the subject not being in need thereof, the method of the invention may be for the prevention of a disease or condition. The subject may be a mammal (e.g., human, mouse, rat, rabbit, dog, cat, cattle, swine, sheep, horse or primate), in and preferably a human. The methods of the invention may be particularly useful for a human subject whom is at least 50, 55, 60, 65, 70, 75, 80, 85, or 90 or more years old.

As used herein, the term “treat,” “treating,” or “treatment” of any disease or disorder may refer to ameliorating the disease or disorder (i.e. slowing or arresting or reducing the development of the disease or at least one of the clinical symptoms thereof). It may also refer to alleviating or ameliorating at least one physical parameter including those which may not be discernible by the patient. It may also refer to modulating the disease or disorder, either physically, (e.g. stabilization of a discernible symptom), physiologically, (e.g. stabilization of a physical parameter), or both. Finally, it may also refer to preventing or delaying the onset or development or progression of the disease or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. Thus, in various aspects, “prevention” of a condition or disorder refers to delaying or preventing the onset of a condition or disorder or reducing its severity, as assessed by the appearance or extent of one or more symptoms of said condition or disorder.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administration” or “administering” mean a route of administration for a compound of Formula (I). Exemplary routes of administration include, but are not limited to, oral, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition. The therapeutic effect may be objective (i.e., measurable by some test or marker) or subjective (i.e., subject gives an indication of or feels an effect).

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians’ Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro- drugs, which become biologically active or more active after they have been placed in a predetermined physiological environment.

As used herein, the term “pulmonary inflammation” includes, but is not limited to, inflammation (more particularly hyperinflammation) of lung tissue, such as that associated with pneumonia and pneumonitis. The inflammation may be caused by cytokine surge, and it includes inflammation which causes ALI and/or ARDS. The cytokine surge is typically viral induced cytokine surge, however it may be caused by non-viral factors, such as autoimmune disease and gas inhalation. The viral-induced cytokine surge may be associated with inflammatory macrophage-monocyte and neutrophil infiltration. Pulmonary inflammation includes inflammation, and particularly hyperinflammation, caused by a coronavirus, i.e., an hCoV. This includes SARS-CoV-2, SARS-CoV, and MERS-CoV.

Without wishing to be bound by theory, VAP-1 (a protein with SSAO activity) may serve as an adhesion molecule, promoting transfer of leukocytes from plasma into inflamed tissues, such as those implemented in pulmonary inflammation. VAP-1’s SSAO activity may enhance local inflammation by releasing small-molecule inflammatory mediators (e.g., hydrogen peroxide, aldehydes and ammonia). VAP-1 may play a role in T-cell driven pro-inflammatory cytokine mediator release (e.g., IFN, IL-1, etc.) contributing to cytokine “storm.” A cytokine storm, including viral-induced cytokine storm, may lead to inflammatory macrophage-monocyte and neutrophil infiltration in lung tissue. This in turn may amplify the cytokine storm, and may be implemented and associated with a hyperinflammation response. The disclosed compounds may suppress unwanted lung mucosal inflammatory cell infiltration and inflammatory cytokine production. This reduces inflammation (or hyperinflammation) in the lung and effectively treats acute lung injury and ARDS.

Additional advantages of the disclosed compounds in the methods of the invention may include a low hERG liability, excellent bioavailability, low to moderate clearance, high permeability (e.g., a PGP substrate leading to low CNS exposure), good in vitro metabolic stability, a suitable half-life (t_{½}) of greater than 5, 6, 7, 8, 9, 10, 11, 12, 13, or 14 hours.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As used herein, the term “one or more” includes 1, 2, 3, 4, 5, or more.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “C₁—C₄ alkyl” refers to a fully saturated branched or unbranched hydrocarbon moiety having from 1 to 4 carbon atoms. Representative examples of C₁—C₄ alkyl include, but are not limited to, methyl, ethyl, n-propyl, iso-propyl, n-butyl, sec-butyl, iso-butyl, and tert-butyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

A “3 to 7-membered cycloalkyl ring” is a saturated or unsaturated non-aromatic ring or ring system, for example a 3, 4, 5, 6, or 7-membered monocyclic, or 6 or 7-membered bicyclic ring. Examples of cycloalkyls include cyclopropyl, cyclobutyl, cyclopentyl, cyclohexyl, cycloheptyl, bicycloheptyl.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or — OA¹—(OA²)ₐ—OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbomenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(—alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(—alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or — C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula —(A¹O(O)C—A²—C(O)O)ₐ— or —(A¹O(O)C—A²—OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula —(A¹O—A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I. In various aspects, these terms can refer to F, Cl, or Br. In various further aspects, these terms can refer to F.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl. In various aspects, the term “heteroaryl,” can refer to a 5- or 6-membered aromatic monocyclic hydrocarbon ring in which at least one (e.g., 1, 2, 3, 4) ring atom is a heteroatom. Examples of five-membered heteroaryls include, but are not limited to, pyrrole, pyrazole, imidazole, triazole, tetrazole, isoxazole, and thiazole. Examples of six-membered heteroaryls include, but are not limited to, pyridinyl, pyrimidinyl, and triazinyl. Thus, in various aspects, when Ar¹ is a heteroaryl, it is a six-membered heteroaryl.

The terms “heterocycle” or “heterocyclyl” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

In various aspects, a “3 to 7-membered heterocyclic ring” refers to a saturated or unsaturated non-aromatic ring or ring system, for example a 3, 4, 5, 6, or 7-membered monocyclic, or 6- or 7-membered bicyclic ring system, and contains at least one heteroatom selected from O, S, and N, where the N and S can also optionally be oxidized to various oxidation states. The heterocyclic ring can be attached at a heteroatom or a carbon atom. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, homomorpholine, and the like.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A3, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)_{0—4}R°; —(CH₂)_{0—4}OR°; —O(CH₂)_{0—4}R°, —O—(CH₂)_{0—4}C(O)OR°; —(CH₂)_{0—4}CH(OR°)₂; —(CH₂)_{0—4}SR°; —(CH₂)_{0—4}Ph, which may be substituted with R°; —(CH₂)_{0—4}O(CH₂)_{0—1}Ph which may be substituted with R°; —CH═CHPh, which may be substituted with R°; —(CH₂)_{0—4}(CH₂)_{0—1}—pyridyl which may be substituted with R°; —NO₂; —CN; —N₃; —(CH₂)_{0—4}N(R°)₂; —(CH₂)_{0—4}N(R°)C(O)R°; —N(R°)C(S)R°; —(CH₂)_{0—4}N(R°)C(O)NR°₂; —N(R°)C(S)NR°₂; —(CH₂)_{0—4}N(R°)C(O)OR°; —N(R°)N(R°)C(O)R°; —N(R°)N(R°)C(O)NR°₂; —N(R°)N(R°)C(O)OR°; —(CH₂)_{0—4}C(O)R°; —C(S)R°; —(CH₂)_{0—4}C(O)OR°; —(CH₂)_{0—4}C(O)SR°; —(CH₂)_{0—4}C(O)OSIR°₃; —(CH₂)_{0—4}OC(O)R°; —OC(O)(CH₂)_{0—4}SR—, SC(S)SR°; —(CH₂)_{0—4}SC(O)R°; —(CH₂)_{0—4}C(O)NR°₂; —C(S)NR°₂; —C(S)SR°; —(CH₂)_{0—4}OC(O)NR°₂; —C(O)N(OR°)R°; —C(O)C(O)R°; —C(O)CH₂C(O)R°; —C(NOR°)R°; —(CH₂)_{0—4}SSR°; —(CH₂)_{0—4}S(O)₂R°; —(CH₂)_{0—4}S(O)₂OR°; —(CH₂)_{0—4}OS(O)₂R°; —S(O)₂NR°₂; —(CH₂)_{0—4}S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°₂; —OP(O)(OR°)₂; SiR°₃; —(C_{1—4} straight or branched alkylene)O—N(R°)₂; or —(C_{1—4} straight or branched alkylene)C(O)O—N(R°)₂, wherein each R° may be substituted as defined below and is independently hydrogen, C₁₋₆ aliphatic, —CH₂Ph, —O(CH₂)_{0—1}Ph, —CH₂—(5—6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), are independently halogen, —(CH₂)_{0—2}R^{•}, —(haloR^{•}), —(CH₂)_{0—2}OH, —(CH₂)_{0—2}OR^{•}, —(CH₂)_{0—}₂CH(OR^{•})₂; —O(haloR^{•}), —CN, —N₃, —(CH₂)_{0—2}C(O)R^{•}, —(CH₂)_{0—}₂C(O)OH, —(CH₂)_{0—}₂C(O)OR^{•}, —(CH₂)_{0—}₂SR^{•}, —(CH₂)_{0—2}SH, —(CH₂)_{0—2}NH₂, —(CH₂)_{0—}₂NHR^{•}, —(CH₂)_{0—}₂NR^{•}₂, —NO₂, —SiR^{•}₃, —OSiR^{•}₃, —C(O)SR^{•}, —(C_{1—4} straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)_{0—1}Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))_{2—3}O—, or —S(C(R*₂))_{2—3}S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)_{2—3}O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, —(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)_{0—1}Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, —(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)_{0—1}Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15 carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15 carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

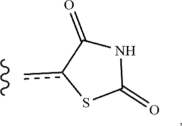

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (-) are employed to designate the sign of rotation of plane-polarized light by the compound, with (-) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

As used herein, the term “prodrugs” refers to compounds that may be converted under physiological conditions or by solvolysis to a biologically active compound of the invention. A prodrug may be inactive when administered to a subject in need thereof, but is converted in vivo to an active compound useful in the methods of the invention. Prodrugs are typically rapidly transformed in vivo to yield a parent compound, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of Formula (I) may be prepared by modifying functional groups, such as a hydroxy, amino, or mercapto groups, present in a compound of Formula (I) in such a way that the modifications are cleaved, either in routine manipulation or in vivo, to the parent compound of Formula (I). Examples of prodrugs include, but are not limited to, acetate, formate and succinate derivatives of hydroxy functional groups or phenyl carbamate derivatives of amino functional groups.

Thus, in various aspects the compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³ H, ¹³ C, ¹⁴ C, ¹⁵ N, ¹⁸ O, ¹⁷ O, ³⁵ S, ¹⁸ F and ³⁶ Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³ H and ¹⁴ C are incorporated, are useful in drug and/or substrate tissue distribution assays. Tritiated, i.e., ³H, and carbon-14, i.e., ¹⁴ C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non- isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an a-hydrogen can exist in an equilibrium of the keto form and the enol form.

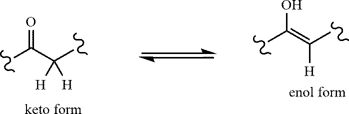

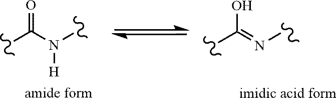

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

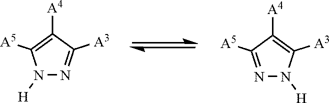

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

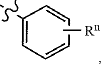

which is understood to be equivalent to a formula:

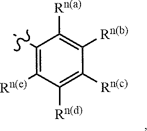

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser’s Reagents for Organic Synthesis, Volumes 1-17 (John Wiley and Sons, 1991); Rodd’s Chemistry of Carbon Compounds, Volumes 1-5 and supplemental volumes (Elsevier Science Publishers, 1989); Organic Reactions, Volumes 1-40 (John Wiley and Sons, 1991); March’s Advanced Organic Chemistry, (John Wiley and Sons, 4th Edition); and Larock’s Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful in treating pulmonary inflammation such as, for example, pulmonary inflammation caused by cytokine surge (e.g., viral induced cytokine surge) or a coronavirus (e.g., severe acute respiratory syndrome coronavirus, severe acute respiratory syndrome coronavirus 2, Middle East respiratory syndrome coronavirus).

In one aspect, the compounds of the invention are useful in the treatment of pulmonary inflammation, as further described herein.

Compounds may be disclosed by the name or chemical structure. If a discrepancy exists between the name of a compound and its associated chemical structure, then the chemical structure prevails.

The disclosed compounds may include isotopically-labelled and/or isotopically-enriched forms of the compounds. The compounds may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl.

The disclosed compounds may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g., arginine and lysine. The term addition salt as used herein also comprises solvates which the compounds and salts thereof are able to form, such as, for example, hydrates, alcoholates, and the like.

Throughout the present disclosure, a given chemical formula or name shall also encompass all pharmaceutically acceptable salts, solvates, hydrates, N-oxides, and/or prodrug forms thereof. It is to be understood that the disclosed compounds include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

The disclosed compounds also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone - enol pairs, amide - imidic acid pairs, lactam - lactim pairs, amide - imidic acid pairs, enamine - imine pairs, and annular forms where a proton can occupy two or more positions of a heterocyclic system, for example, 1H- and 3H-imidazole, 1H, 2H- and 4H- 1,2,4-triazole, 1H- and 2H- isoindole, and 1H- and 2H-pyrazole. Tautomeric forms can be in equilibrium or sterically locked into one form by appropriate substitution.

The disclosed compounds can be asymmetric (e.g. having one or more stereocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C=N double bonds, and the like can also be present in the compounds described herein, and all such stable isomers are contemplated in the present invention. Cis- and trans-geometric isomers of the compounds are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those disclosed compounds that contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The disclosed compounds may be formulated into pharmaceutical compositions (or formulations) for various modes of administration. It will be appreciated that compounds may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration. Other formulations may conveniently be presented in unit dosage form, e.g. tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow release formulations.

The disclosed compounds are preferably administered in the methods of the invention in a pharmaceutical composition.

The dose level and frequency of dosage of the specific compound will vary depending on a variety of factors including the potency of the specific compound employed, the metabolic stability and length of action of that compound, the patient’s age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g., from about 0.01 mg to about 25 mg each. Normally, such a dosage is given orally but parenteral administration may also be chosen.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds of Formula (I), or a pharmaceutically acceptable salt, solvate, hydrate, N-oxide, and/or prodrug thereof:

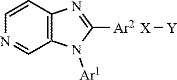

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo or C1-C4 alkyl groups; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

In one aspect, disclosed are compounds of Formula (I), or a pharmaceutically acceptable salt, solvate, hydrate, N-oxide, and/or prodrug thereof:

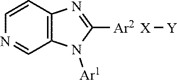

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

In a further aspect, the compound of Formula (I) may be a compound of Formula (I′):

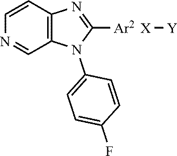

In a further aspect, the compound of Formula (I) may be a compound of Formula (I’’):

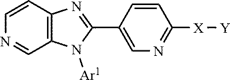

In a further aspect, the compound of Formula (I) is a compound of Formula (I‴):

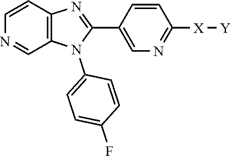

In a further aspect, the compound of Formula (I) is a compound for Formula (I⁗):

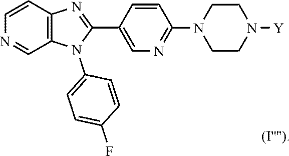

In a further aspect, disclosed are compounds having a structure represented by a formula:

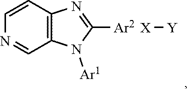

wherein Ar¹ is phenyl or a 5- or 6-membered heteroaryl, and is substituted with 0, 1, 2, or 3 halogen groups; wherein Ar² is phenyl or a 5- or 6-membered heteroaryl, and is substituted with 0, 1, 2, or 3 halogen groups; wherein X is a 3- to 7-membered heterocycloalkyl or a 3- to 7-membered cycloalkyl, and is substituted with 0, 1, 2, or 3 groups selected from halogen and C1-C4 alkyl; wherein Y is selected from the group consisting of C1-C4 alkyl, C1-C4 haloalkyl, —CN, —OR¹, —C(O)R¹, —NR²R³, —NR¹C(O)R⁴, —C(O)NR²R³, —S(O)R¹, —SO₂R¹, —S(O)NR²R³, -and SO₂NR²R³; wherein R¹ is selected from the group consisting of H, C1-C4 alkyl, and C1-C4 haloalkyl; wherein each of R² and R³ are independently selected from the group consisting of H, C1-C4 alkyl, and C1-C4 haloalkyl, or wherein each of R² and R³ together with the nitrogen to which they are attached comprise a 3- to 7-membered heterocycloalkyl substituted with 0, 1, 2, or 3 halogen groups; and wherein R⁴ is selected from the group consisting of H, C1-C4 alkyl, and C1-C4 haloalkyl, or a pharmaceutically acceptable salt thereof.

In a further aspect, the compound has a structure represented by a formula:

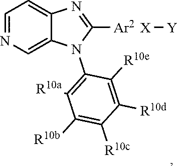

wherein each of R^{10a}, R^{10b}, R^{10c}, R^{10d}, and R^{10e} is independently selected from hydrogen and halogen, provided that at least two of R^{10a}, R^{10b}, R^{10c}, R^{10d}, and R^{10e} are hydrogen.

In a further aspect, the compound has a structure represented by a formula:

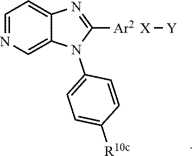

In a further aspect, the compound has a structure represented by a formula:

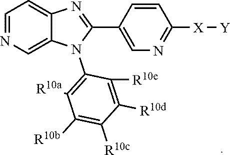

In a further aspect, the compound has a structure represented by a formula:

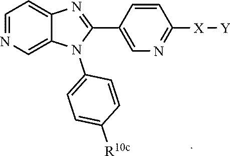

In a further aspect, the compound has a structure represented by a formula:

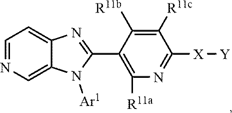

wherein each of R^{11a}, R^{11b}, and R^{11c} is independently selected from hydrogen and halogen.

In a further aspect, the compound has a structure represented by a formula:

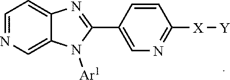

In a further aspect, the compound has a structure represented by a formula:

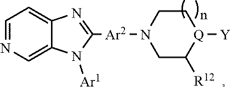

wherein n is 1 or 2; wherein Q is O, N, or CH; and wherein R¹² is H, halogen, or C1-C4 alkyl.

In a further aspect, the compound has a structure represented by a formula:

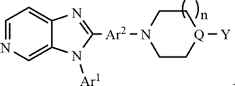

In a further aspect, the compound has a structure represented by a formula:

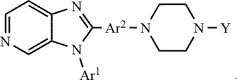

In one aspect, n is 1 or 2. In a further aspect, n is 1. In a still further aspect, n is 2.

In a further aspect, the compound of Formula (I) is 1-{5-[3-(4-fluorophenyl)-3H-imidazo[4,5-c]pyridin-2-yl]pyridin-2-yl}-4-methanesulfonylpiperazine, or a pharmaceutically acceptable salt, solvate, hydrate, N-oxide, and/or prodrug thereof:

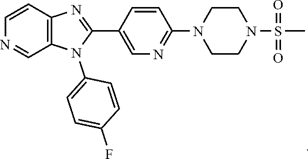

### A. Q Groups

In one aspect, Q is O, N, or CH. In a further aspect, Q is N or CH. In a still further aspect, Q is O or CH. In yet a further aspect, Q is O or N. In an even further aspect, Q is O. In a still further aspect, Q is N. In yet a further aspect, Q is CH.

### B. X Groups

In one aspect, X is selected from the group consisting of 3- to 7-membered heterocyclic ring and 3- to 7-membered cycloalkyl ring, each of which is optionally substituted with one or more halo. In a further aspect, X is a 3- to 7-membered heterocycloalkyl or a 3- to 7-membered cycloalkyl, and is substituted with 0, 1, 2, or 3 halogen groups. In a still further aspect, X is a 3- to 7-membered heterocycloalkyl or a 3- to 7-membered cycloalkyl, and is substituted with 0, 1, or 2 halogen groups. In yet a further aspect, X is a 3- to 7-membered heterocycloalkyl or a 3- to 7-membered cycloalkyl, and is substituted with 0 or 1 halogen group. In an even further aspect, X is a 3- to 7-membered heterocycloalkyl or a 3- to 7-membered cycloalkyl, and is monosubstituted with a halogen group. In a still further aspect, X is a 3- to 7-membered heterocycloalkyl or a 3- to 7-membered cycloalkyl, and is unsubstituted.

In various aspects, group X may be selected from the group consisting of 3 to 7-membered heterocyclic ring and 3 to 7-membered cycloalkyl ring, each of which is optionally substituted with one or more halo.

In various aspects, group X is a 3 to 7-membered heterocyclic ring, optionally substituted with one or more halo. In various further aspects, it is a 6-membered heterocyclic ring optionally substituted with one or more halo. In various further aspects, it is a 6-membered heterocyclic ring (e.g., a piperazine) optionally substituted with one or more halo. In various further aspects, group X is of Formula (B) piperazine:

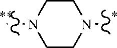

In various aspects, in Formula (B), the bond marked * is directly connected to group Y, and the bond marked ** is directly connected to Ar². Thus, in various aspects, the compound of Formula (I) may be a compound for Formula (I⁗):

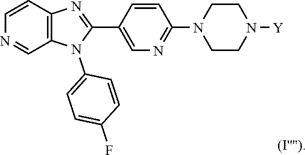

In various aspects, X is a 3- to 7-membered heterocyclic ring optionally substituted with one or more halo. Examples of 3- to 7-membered heterocyclic rings include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In a further aspect, X is a 3- to 7-membered heterocycloalkyl substituted with 0, 1, 2, or 3 halogen groups. In a still further aspect, X is a 3- to 7-membered heterocycloalkyl substituted with 0, 1, or 2 halogen groups. In yet a further aspect, X is a 3-to 7-membered heterocycloalkyl substituted with 0 or 1 halogen group. In an even further aspect, X is a 3- to 7-membered heterocycloalkyl monosubstituted with a halogen group. In a still further aspect, X is an unsubstituted 3- to 7-membered heterocycloalkyl.

In various aspects, X is a 6-membered heterocyclic ring optionally substituted with one or more halo. In a further aspect, X is a 6-membered heterocycloalkyl substituted with 0, 1, 2, or 3 halogen groups. In a still further aspect, X is a 6-membered heterocycloalkyl substituted with 0, 1, or 2 halogen groups. In yet a further aspect, X is a 6-membered heterocycloalkyl substituted with 0 or 1 halogen group. In an even further aspect, X is a 6-membered heterocycloalkyl monosubstituted with a halogen group. In a still further aspect, X is an unsubstituted 6-membered heterocycloalkyl.

In various aspects, X is a a 6-membered heterocyclic ring of Formula (B):

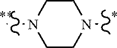

wherein the bond marked * is directly connected to B, and the bond marked ** is directly connected to Ar².

In various aspects, X is a 3- to 7-membered cycloalkyl ring optionally substituted with one or more halo. Examples of 3- to 7-membered cycloalkyl rings include, but are not limited to, cyclopropyl, cyclobutyl, cyclopentyl, and cyclohexyl. In a further aspect, X is a 3-to 7-membered cycloalkyl substituted with 0, 1, 2, or 3 halogen groups. In a still further aspect, X is a 3- to 7-membered cycloalkyl substituted with 0, 1, or 2 halogen groups. In yet a further aspect, X is a 3- to 7-membered cycloalkyl substituted with 0 or 1 halogen group. In an even further aspect, X is a 3- to 7-membered cycloalkyl monosubstituted with a halogen group. In a still further aspect, X is an unsubstituted 3- to 7-membered cycloalkyl.

### C. Y Groups

In one aspect, Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR2R3, —SO₂NR²R³, wherein the C₁—C₄ alkyl is optionally substituted with one or more halo. In a further aspect, Y is selected from the group consisting of C1-C4 alkyl, C1-C4 haloalkyl, —CN, —OR¹, —C(O)R¹, —NR²R³, —NR¹C(O)R⁴, —C(O)NR²R³, —S(O)R¹, —SO₂R¹, —S(O)NR²R³, and —SO₂NR²R³. In a still further aspect, Y is selected from the group consisting of methyl, ethyl, n-propyl, isopropyl, —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, —CH(CH₃)CH₂Cl, —CN, —OR¹, —C(O)R¹, —NR²R³, —NR¹C(O)R⁴, —C(O)NR²R³, —S(O)R¹, —SO₂R¹, —S(O)NR²R³, and —SO₂NR²R³. In yet a further aspect, Y is selected from the group consisting of methyl, ethyl, —CH₂F, —CHF₂, —CF₃, —CH₂CH₂F, —CH₂Cl, —CHCl₂, —CCl₃, —CH₂CH₂Cl, —CN, —OR¹, —C(O)R¹, —NR²R³, —NR¹C(O)R⁴, —C(O)NR²R³, —S(O)R¹, —SO₂R¹, —S(O)NR²R³, and —SO₂NR²R³. In an even further aspect, Y is selected from the group consisting of methyl, —CH₂F, —CHF₂, —CF₃, —CH₂Cl, —CHCl₂, —CCl₃, —CN, —OR¹, —C(O)R¹, —NR²R³, —NR¹C(O)R⁴, —C(O)NR2R3, —S(O)R¹, —SO₂R¹, —S(O)NR²R³, and —SO₂NR²R³.

In various aspects, group Y may be selected from the group consisting of C₁—C₄ alkyl (optionally substituted with one or more halo), —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³. R¹, R², R³, and R⁴ are as defined elsewhere herein.

In various aspects, Y is C₁—C₄ alkyl (optionally substituted with one or more halo), —OR¹, —C(O)R¹, —NR²R³, —NR¹C(O)R⁴, —C(O)NR2R3, —SO₂R¹. In various further aspects, Y is —SO₂R¹. In various further aspects, R¹ is methyl, making Y the group —SO₂Me.

In various aspects, Y is selected from the group consisting of C1-C4 alkyl and Cl-C4 haloalkyl. In a further aspect, Y is selected from the group consisting of methyl, ethyl, n-propyl, isopropyl, —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In a still further aspect, Y is selected from the group consisting of methyl, ethyl, —CH₂F, —CHF₂, —CF₃, —CH₂CH₂F, —CH₂Cl, —CHCl₂, —CCl₃, and —CH₂CH₂Cl. In yet a further aspect, Y is selected from the group consisting of methyl, —CH₂F, —CHF₂, —CF₃, —CH₂Cl, —CHCl₂, and —CCl₃.

In various aspects, Y is selected from the group consisting of —CN, —OR¹, —C(O)R¹, and —C(O)NR²R³. In a further aspect, Y is selected from the group consisting of —CN, —C(O)R¹, and —C(O)NR²R³. In a still further aspect, Y is selected from the group consisting of —C(O)R¹ and —C(O)NR²R³. In yet a further aspect, Y is —C(O)R¹. In an even further aspect, Y is —C(O)NR²R³. In a still further aspect, Y is —OR¹. In yet a further aspect, Y is —CN.

In various aspects, Y is selected from the group consisting of —NR²R³ and —NR¹C(O)R⁴. In a further aspect, Y is —NR²R³. In a still further aspect, Y is —NR¹C(O)R⁴.

In various aspects, Y is selected from the group consisting of —S(O)R¹, —SO₂R¹, —S(O)NR²R³, and —SO₂NR²R³. In a further aspect, Y is selected from the group consisting of —S(O)R¹ and —S(O)NR²R³. In a still further aspect, Y is selected from the group consisting of —SO₂R¹ and —SO₂NR²R³. In yet a further aspect, Y is —S(O)R¹. In an even further aspect, Y is —S(O)NR²R³. In a still further aspect, Y is —SO₂NR²R³. In yet a further aspect, Y is —SO₂R¹. In an even further aspect, Y is —SO₂Me.

### D. R¹ Groups

In one aspect, R¹ is selected from the group consisting of H and C₁—C₄ alkyl, wherein the C₁—C₄ alkyl is optionally substituted with one or more halo. In a further aspect, R¹ is selected from the group consisting of H, C1-C4 alkyl, and C1-C4 haloalkyl. In a still further aspect, R¹ is selected from the group consisting of H, methyl, ethyl, n-propyl, isopropyl, —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In yet a further aspect, R¹ is selected from the group consisting of H, methyl, ethyl, —CH₂F, —CHF₂, —CF₃, —CH₂CH₂F, —CH₂Cl, —CHCl₂, —CCl₃, and —CH₂CH₂Cl. In an even further aspect, R¹ is selected from the group consisting of H, methyl, —CH₂F, —CHF₂, —CF₃, —CH₂Cl, —CHCl₂, and —CCl₃.

In various aspects, R¹ is selected from the group consisting of H and C₁—C₄ alkyl. In a still further aspect, R¹ is selected from the group consisting of H, methyl, ethyl, n-propyl, and isopropyl. In yet a further aspect, R¹ is selected from the group consisting of H, methyl, and ethyl. In an even further aspect, R¹ is selected from the group consisting of H and methyl.

In various aspects, R¹ is C₁—C₄ alkyl. In a still further aspect, R¹ is selected from the group consisting of methyl, ethyl, n-propyl, and isopropyl. In yet a further aspect, R¹ is selected from the group consisting of methyl and ethyl. In an even further aspect, R¹ is ethyl. In a still further aspect, R¹ is methyl.

In various aspects, R¹ is selected from the group consisting of H and C₁—C₄ haloalkyl. In a still further aspect, R¹ is selected from the group consisting of H, —CH₂F, —CHF₂, —CF₃, —CH₂CH₂F, —CH₂CH₂CH₂F, —CH(CH₃)CH₂F, —CH₂Cl, —CHCl₂, —CCl₃,—CH₂CH₂Cl, —CH₂CH₂CH₂Cl, and —CH(CH₃)CH₂Cl. In yet a further aspect, R¹ is selected from the group consisting of H, —CH₂F, —CHF₂, —CF₃, —CH₂CH₂F, —CH₂Cl, —CHCl₂, —CCl₃, and —CH₂CH₂Cl. In an even further aspect, R¹ is selected from the group consisting of H, —CH₂F, —CHF₂, —CF₃, —CH₂Cl, —CHCl₂, and —CCl₃.

In various aspects, R¹ is C1-C4 haloalkyl. In a still further aspect, R¹ is selected from the group consisting of —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In yet a further aspect, R¹ is selected from the group consisting of —CH₂F, —CHF₂, —CF₃, —CH₂CH₂F, —CH₂Cl, —CHCl₂, —CCl₃, and —CH₂CH₂Cl. In an even further aspect, R¹ is selected from the group consisting of —CH₂F, —CHF₂, —CF₃, —CH₂Cl, —CHCl₂, and —CCl₃. In a still further aspect, R¹ is selected from the group consisting of —CH₂F, —CHF₂, —CH₂Cl, and —CHCl₂. In yet a further aspect, R¹ is selected from the group consisting of —CH₂F and —CH₂Cl.

In various aspects, R¹ is H.

### E. R² and R³ Groups

In one aspect, R² and R³ are each independently selected from the group consisting of H and C₁—C₄ alkyl, wherein the C₁—C₄ alkyl is optionally substituted with one or more halo or R² and R³ are taken together with the nitrogen to which they are attached to form a 3- to 7-membered heterocyclic ring optionally substituted with one or more halo. In a further aspect, each of R² and R³ are independently selected from the group consisting of H, C₁—C₄ alkyl, and C1-C4 haloalkyl, or each of R² and R³ together with the nitrogen atom to which they are attached comprise a 3- to 7-membered heterocycloalkyl substituted with 0, 1, 2, or 3 halogen groups.

In various aspects, each of R² and R³ are independently selected from the group consisting of H, C₁—C₄ alkyl, and C₁—C₄ haloalkyl. In a further aspect, each of R² and R³ are independently selected from the group consisting of H, methyl, ethyl, n-propyl, isopropyl, —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In a still further aspect, each of R² and R³ are independently selected from the group consisting of H, methyl, ethyl, —CH₂F, —CHF₂, —CF₃, —CH₂CH₂F, —CH₂Cl, —CHCl₂, —CCl₃, and —CH₂CH₂Cl. In yet a further aspect, each of R²and R³ are independently selected from the group consisting of H, methyl, —CH₂F, —CHF₂, —CF₃, —CH₂Cl, —CHCl₂, and —CCl₃.

In various aspects, each of R² and R³ are independently selected from the group consisting of H and C₁—C₄ alkyl. In a further aspect, each of R² and R³ are independently selected from the group consisting of H, methyl, ethyl, n-propyl, and isopropyl. In a still further aspect, each of R² and R³ are independently selected from the group consisting of H, methyl, and ethyl. In yet a further aspect, each of R² and R³ are independently selected from the group consisting of H and methyl.

In various aspects, each of R² and R³ are independently C1-C4 alkyl. In a further aspect, each of R² and R³ are independently selected from the group consisting of methyl, ethyl, n-propyl, and isopropyl. In a still further aspect, each of R² and R³ are independently selected from the group consisting of methyl and ethyl. In yet a further aspect, each of R² and R³ are ethyl. In an even further aspect, each of R² and R³ are methyl.

In various aspects, each of R² and R³ are independently selected from the group consisting of H and C1-C4 haloalkyl. In a further aspect, each of R² and R³ are independently selected from the group consisting of H, —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In a still further aspect, each of R² and R³ are independently selected from the group consisting of H, —CH₂F, —CHF₂, —CF₃, —CH₂CH₂F, —CH₂Cl, —CHCl₂, —CCl₃, and —CH₂CH₂Cl. In yet a further aspect, each of R² and R³ are independently selected from the group consisting of H, —CH₂F, —CHF₂, —CF₃, —CH₂Cl, —CHCl₂, and —CCl₃.

In various aspects, each of R² and R³ are C1-C4 haloalkyl. In a further aspect, each of R² and R³ are independently selected from the group consisting of —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In a still further aspect, each of R² and R³ are independently selected from the group consisting of —CH₂F, —CHF₂, —CF₃, —CH₂CH₂F, —CH₂Cl, —CHCl₂, —CCl₃, and —CH₂CH₂Cl. In yet a further aspect, each of R² and R³ are independently selected from the group consisting of —CH₂F, —CHF₂, —CF₃, —CH₂Cl, —CHCl₂, and —CCl₃.

In various aspects, each of R² and R³ are H.

In various aspects, each of R² and R³ together with the nitrogen atom to which they are attached comprise a 3- to 7-membered heterocycloalkyl substituted with 0, 1, 2, or 3 halogen groups. Examples of 3- to 7-membered heterocycloalkyls include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In a further aspect, each of R² and R³ together with the nitrogen atom to which they are attached comprise a 3- to 7-membered heterocycloalkyl substituted with 0, 1, or 2 halogen groups. In a still further aspect, each of R² and R³ together with the nitrogen atom to which they are attached comprise a 3- to 7-membered heterocycloalkyl substituted with 0 or 1 halogen group. In yet a further aspect, each of R² and R³ together with the nitrogen atom to which they are attached comprise a 3- to 7-membered heterocycloalkyl monosubstituted with a halogen group. In an even further aspect, each of R² and R³ together with the nitrogen atom to which they are attached comprise an unsubstituted 3- to 7-membered heterocycloalkyl.

When R² and R³ are taken together with the nitrogen to which they are attached to form a 3 to 7-membered heterocyclic ring (which may optionally be substituted with one or more halo), the heterocyclic ring may be those defined elsewhere herein, with the proviso that at least one nitrogen atom is present. Examples of 3 to 7-membered heterocyclic rings include, but are not limited to, pyrrolidine and piperidine.

### F. R⁴ Groups

In one aspect, R⁴ is selected from the group consisting of H and C₁—C₄ alkyl, wherein the C₁—C₄ alkyl is optionally substituted with one or more halo. In a further aspect, R⁴ is selected from the group consisting of H, C1-C4 alkyl, and C1-C4 haloalkyl. In a still further aspect, R⁴ is selected from the group consisting of H, methyl, ethyl, n-propyl, isopropyl, —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In yet a further aspect, R⁴ is selected from the group consisting of H, methyl, ethyl, —CH₂F, —CHF₂, —CF₃, —CH₂CH₂F, —CH₂Cl, —CHCl₂, —CCl₃, and —CH₂CH₂Cl. In an even further aspect, R⁴ is selected from the group consisting of H, methyl, —CH₂F, —CHF₂, —CF₃, —CH₂Cl, —CHCl₂, and —CCl₃.

In various aspects, R⁴ is selected from the group consisting of H and C1-C4 alkyl. In a still further aspect, R⁴ is selected from the group consisting of H, methyl, ethyl, n-propyl, and isopropyl. In yet a further aspect, R⁴ is selected from the group consisting of H, methyl, and ethyl. In an even further aspect, R⁴ is selected from the group consisting of H and methyl.

In various aspects, R⁴ is C1-C4 alkyl. In a still further aspect, R⁴ is selected from the group consisting of methyl, ethyl, n-propyl, and isopropyl. In yet a further aspect, R⁴ is selected from the group consisting of methyl and ethyl. In an even further aspect, R⁴ is ethyl. In a still further aspect, R⁴ is methyl.

In various aspects, R⁴ is selected from the group consisting of H and C1-C4 haloalkyl. In a still further aspect, R⁴ is selected from the group consisting of H, —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In yet a further aspect, R⁴ is selected from the group consisting of H, —CH₂F, —CHF₂, —CF₃, —CH₂CH₂F, —CH₂Cl, —CHCl₂, —CCl₃, and —CH₂CH₂Cl. In an even further aspect, R⁴ is selected from the group consisting of H, —CH₂F, —CHF₂, —CF₃, —CH₂Cl, —CHCl₂, and —CCl₃.

In various aspects, R⁴ is C1-C4 haloalkyl. In a still further aspect, R⁴ is selected from the group consisting of —CH₂F, —CHF₂, —CF₃, —CH₂CH₂F, —CH₂CH₂CH₂F, —CH(CH₃)CH₂F, —CH₂Cl, —CHCl₂, —CCl₃, —CH₂CH₂Cl, —CH₂CH₂CH₂Cl, and —CH(CH₃)CH₂Cl. In yet a further aspect, R⁴ is selected from the group consisting of —CH₂F, —CHF₂, —CF₃, —CH₂CH₂F, —CH₂Cl, —CHCl₂, —CCl₃, and —CH₂CH₂Cl. In an even further aspect, R⁴ is selected from the group consisting of —CH₂F, —CHF₂, —CF₃, —CH₂Cl, —CHCl₂, and —CCl₃. In a still further aspect, R⁴ is selected from the group consisting of —CH₂F, —CHF₂, —CH₂Cl, and —CHCl₂. In yet a further aspect, R⁴ is selected from the group consisting of —CH₂F and —CH₂Cl.

In various aspects, R⁴ is H.

### G. R^{10A}, R^{10B}, R^{10C}, R^{10D,} AND R^{10E} Groups

In one aspect, each of R^{10a}, R^{10b}, R^{10c}, R^{10d}, and R^{10e} is independently selected from hydrogen and halogen, provided that at least two of R^{10a}, R^{10b}, R^{10c}, R^{10d}, and R^{10e} are hydrogen. In a further aspect, each of R^{10a}, R^{10b}, R^{10c}, R^{10d}, and R^{10e} is independently selected from hydrogen, —F, —Cl, and —Br. In a still further aspect, each of R^{10a}, R^{10b}, R^{10c}, R^{10d}, and R^{10e} is independently selected from hydrogen, —F, and —Cl. In yet a further aspect, each of R^{10a}, R^{10b}, R^{10c}, R^{10d}, and R^{10e} is independently selected from hydrogen and —Cl. In an even further aspect, each of R^{10a}, R^{10b}, R^{10c}, R^{10d}, and R^{10e} is independently selected from hydrogen and —F.

In various aspects, at least two of R^{10a}, R^{10b}, R^{10c}, R^{10d}, and R^{10e} are hydrogen. In a further aspect, at least three of R^{10a}, R^{10b}, R^{10c}, R^{10d}, and R^{10e} are hydrogen. In a still further aspect, at least four of R^{10a}, R^{10b}, R^{10c}, R^{10d}, and R^{10e} are hydrogen. In yet a further aspect, at least three of R^{10a}, R^{10b}, R^{10c}, R^{10d}, and R^{10e} are halogen. In an even further aspect, at least two of R^{10a}, R^{10b}, R^{10c}, R^{10d}, and R^{10e} are halogen. In a still further aspect, one of R^{10a}, R^{10b}, R^{10c}, R^{10d}, and R^{10e} are hydrogen.

In various aspects, each of R^{10a}, R^{10b}, R^{10d}, and R^{10e} is hydrogen and R^{10c} is halogen. In a further aspect, each of R^{10a}, R^{10b}, R^{10d}, and R^{10e} is hydrogen and R^{10c} is —F, —Cl, or —Br. In a still further aspect, each of R^{10a}, R^{10b}, R^{10d}, and R^{10e} is hydrogen and R^{10c} is —F or —Cl.

In various aspects, each of R^{10a}, R^{10b}, R^{10c}, R^{10d}, and R^{10e} is hydrogen.

### H. R Groups

In one aspect, each of R^{11a}, R^{11b}, and R^{11c} is independently selected from hydrogen and halogen. In a further aspect, each of R^{11a}, R^{11b}, and R^{11c} is independently selected from hydrogen, —F, —Cl, and —Br. In a still further aspect, ach of R^{11a}, R^{11b}, and R^{11c} is independently selected from hydrogen, —F, and —Cl. In yet a further aspect, ach of R^{11a}, R^{11b}, and R^{11c} is independently selected from hydrogen and —Cl. In an even further aspect, ach of R^{11a}, R^{11b}, and R^{11C} is independently selected from hydrogen and —F.

In various aspects, each of R^{11a}, R^{11b}, and R^{11c} is hydrogen.

### I. R¹² Groups

In one aspect, R¹² is H, halogen, or C1-C4 alkyl. In a further aspect, R¹² is H, —F, —Cl, —Br, methyl, ethyl, n-propyl, or isopropyl. In a still further aspect, R¹² is H, —F, —Cl, —Br, methyl, or ethyl. In yet a further aspect, R¹² is H, —F, —Cl, —Br, or methyl.

In various aspects, R¹² is H or C1-C4 alkyl. In a further aspect, R¹² is H, methyl, ethyl, n-propyl, or isopropyl. In a still further aspect, R¹² is H, methyl, or ethyl. In yet a further aspect, R¹² is H or methyl.

In various aspects, R¹² is H or halogen. In a further aspect, R¹² is H, —F, —Cl, or —Br. In a still further aspect, R¹² is H, —F, or —Cl. In yet a further aspect, R¹² is H or —Cl. In an even further aspect, R¹² is H or —F.

In various aspects, R¹² is H.

### J. AR¹ Groups

In one aspect, Ar¹ is selected from the group consisting of phenyl and heteroaryl, each of which is optionally substituted with one or more halo. In a further aspect, Ar¹ is phenyl or 5- or 6-membered heteroaryl, and is substituted with 0, 1, 2, or 3 halogen groups. In a still further aspect, Ar¹ is phenyl or 5- or 6-membered heteroaryl, and is substituted with 0, 1, or 2 halogen groups. In yet a further aspect, Ar¹ is phenyl or 5- or 6-membered heteroaryl, and is substituted with 0 or 1 halogen group. In an even further aspect, Ar¹ is phenyl or 5- or 6-membered heteroaryl, and is monosubstituted with a halogen group. In a still further aspect, Ar¹ is phenyl or 5- or 6-membered heteroaryl, and is unsubstituted.

In various aspects, Ar¹ may be selected from the group consisting of phenyl and heteroaryl. Each of phenyl and heteroaryl may be substituted with one or more halo atoms.

Without wishing to be bound by theory, in various aspects, Ar¹ is phenyl that is optionally substituted with one or more halo, such as one or more F. In various further aspects, Ar¹ is phenyl optionally substituted with one F. In various further aspects, Ar¹ is 4-fluoro phenyl. Thus, in various aspects, the compound of Formula (I) may be a compound of Formula (I′):

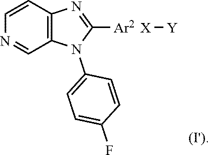

In various aspects, Ar¹ is phenyl optionally substituted with one or more halo. In a further aspect, Ar¹ is phenyl substituted with 0, 1, 2, or 3 halogen groups. In a still further aspect, Ar¹ is phenyl substituted with 0, 1, or 2 halogen groups. In yet a further aspect, Ar¹ is phenyl substituted with 0 or 1 halogen group. In an even further aspect, Ar¹ is phenyl monosubstituted with a halogen group. In a still further aspect, Ar¹ is unsubstituted phenyl.

In various aspects, Ar¹ is phenyl optionally substituted with one or more fluorines. In a further aspect, Ar¹ is phenyl substituted with 0, 1, 2, or 3 fluorine groups. In a still further aspect, Ar¹ is phenyl substituted with 0, 1, or 2 fluorine groups. In yet a further aspect, Ar¹ is phenyl substituted with 0 or 1 fluorine group. In an even further aspect, Ar¹ is phenyl monosubstituted with a fluorine group. In a still further aspect, Ar¹ is 4-fluoro-phenyl.

In various aspects, Ar¹ is heteroaryl optionally substituted with one or more halo. Examples of heteroaryls include, but are not limited to, furanyl, thiophenyl, pyrrolyl, benzo[d]thiazole, pyrazolyl, imidazolyl, triazolyl, oxazolyl, thiazolyl, pyridinyl, pyrimidinyl, imidazolyl, purinyl, indolyl, and quinolinyl. In a further aspect, Ar¹ is 5- or 6-membered heteroaryl substituted with 0, 1, 2, or 3 halogen groups. In a still further aspect, Ar¹ is 5- or 6-membered heteroaryl substituted with 0, 1, or 2 halogen groups. In yet a further aspect, Ar¹ is 5- or 6-membered heteroaryl substituted with 0 or 1 halogen group. In an even further aspect, Ar¹ is 5- or 6-membered heteroaryl monosubstituted with a halogen group. In a still further aspect, Ar¹ is unsubstituted 5- or 6-membered heteroaryl.

### K. AR² Groups

In one aspect, Ar² is selected from the group consisting of phenyl and heteroaryl, each of which is optionally substituted with one or more halo. In a further aspect, Ar² is phenyl or 5- or 6-membered heteroaryl, and is substituted with 0, 1, 2, or 3 halogen groups. In a still further aspect, Ar² is phenyl or 5- or 6-membered heteroaryl, and is substituted with 0, 1, or 2 halogen groups. In yet a further aspect, Ar² is phenyl or 5- or 6-membered heteroaryl, and is substituted with 0 or 1 halogen group. In an even further aspect, Ar² is phenyl or 5- or 6-membered heteroaryl, and is monosubstituted with a halogen group. In a still further aspect, Ar² is phenyl or 5- or 6-membered heteroaryl, and is unsubstituted.

In the methods of the invention, Ar² is selected from the group consisting of phenyl and heteroaryl, each of which is optionally substituted with one or more halo. Ar² may be selected from pyridinyl, pyrimidinyl and pyrazine each of which is optionally substituted with one or more halo.

In various aspects, “pyridinyl” is a divalent pyridine radical, “pyrimidinyl” is a divalent pyrimidine radical, and “pyrazine” is a divalent pyrazine radical. Substitution patterns for pyridinyl, pyrimidinyl and pyrazine include, but are not limited to, the following.

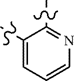

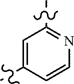

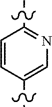

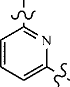

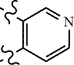

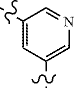

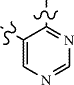

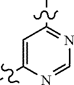

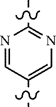

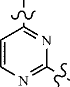

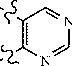

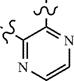

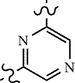

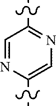

Without wishing to be bound by theory, in various aspects, Ar² is a pyridinyl. In various further aspects, Ar² is a pyridinyl of Formula (A):

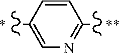

In various aspects, in Formula (A), the bond marked * is directly connected to the imidazo[4,5-c]pyridine, and the bond marked ** is directly connected to X in the compound of Formula (I). This forms a compound for Formula (I”):

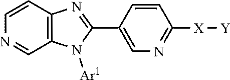

In various aspects, Ar² is phenyl optionally substituted with one or more halo. In a further aspect, Ar² is phenyl substituted with 0, 1, 2, or 3 halogen groups. In a still further aspect, Ar² is phenyl substituted with 0, 1, or 2 halogen groups. In yet a further aspect, Ar² is phenyl substituted with 0 or 1 halogen group. In an even further aspect, Ar² is phenyl monosubstituted with a halogen group. In a still further aspect, Ar² is unsubstituted phenyl.

In various aspects, Ar² is heteroaryl optionally substituted with one or more halo. Examples of heteroaryls include, but are not limited to, furanyl, thiophenyl, pyrrolyl, benzo[d]thiazole, pyrazolyl, imidazolyl, triazolyl, oxazolyl, thiazolyl, pyridinyl, pyrimidinyl, imidazolyl, purinyl, indolyl, and quinolinyl. In a further aspect, Ar² is 5- or 6-membered heteroaryl substituted with 0, 1, 2, or 3 halogen groups. In a still further aspect, Ar² is 5- or 6-membered heteroaryl substituted with 0, 1, or 2 halogen groups. In yet a further aspect, Ar² is 5- or 6-membered heteroaryl substituted with 0 or 1 halogen group. In an even further aspect, Ar² is 5- or 6-membered heteroaryl monosubstituted with a halogen group. In a still further aspect, Ar² is unsubstituted 5- or 6-membered heteroaryl.

In various aspects, Ar² is pyridinyl or pyrimidinyl optionally substituted with one or more halo. In a further aspect, Ar² is pyridinyl or pyrimidinyl, and is substituted with 0, 1, 2, or 3 halogen groups. In a still further aspect, Ar² is pyridinyl or pyrimidinyl, and is substituted with 0, 1, or 2 halogen groups. In yet a further aspect, Ar² is pyridinyl or pyrimidinyl, and is substituted with 0 or 1 halogen group. In an even further aspect, Ar² is pyridinyl or pyrimidinyl, and is monosubstituted with a halogen group. In a still further aspect, Ar² is pyridinyl or pyrimidinyl, and is unsubstituted.

In various aspects, Ar² is pyridinyl.

In various aspects, Ar² is a pyridinyl of Formula (A):

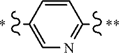

wherein the bond marked * is directly connected to the imidazo[4,5-c]pyridine, and the bond marked ** is directly connected to X.

### 2. Example Compounds

In one aspect, a compound can be present as:

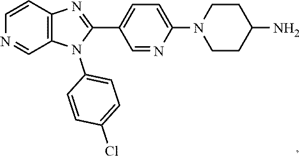

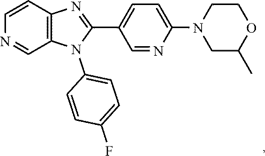

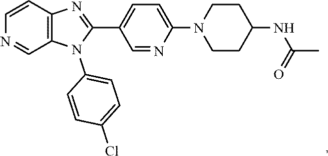

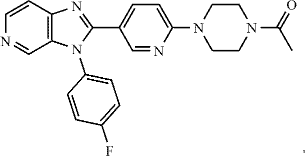

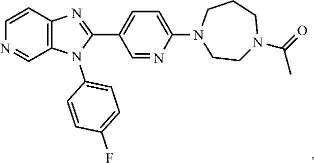

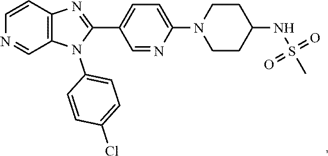

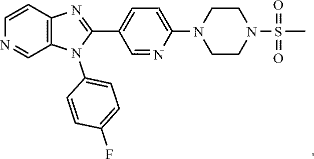

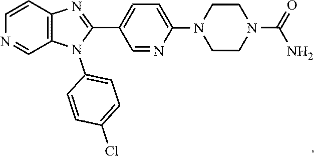

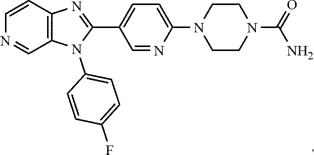

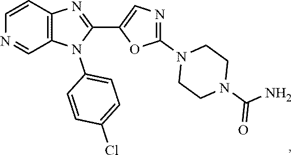

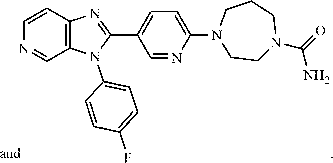

or a pharmaceutically acceptable salt thereof.

In one aspect, a compound can be present as:

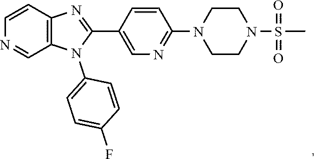

or a pharmaceutically acceptable salt thereof.

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. Methods of Making a Compound

The disclosed compounds may be prepared by, or in analogy with, conventional methods. The preparation of intermediates and compounds according to the examples of the present invention may in particular be illuminated by the following Schemes. Definitions of variables in the structures in schemes herein are commensurate with those of corresponding positions in the formulas delineated herein.

SCHEME 1. GENERAL SYNTHETIC ROUTES FOR PREPARATION OF COMPOUNDS OF FORMULA (I)

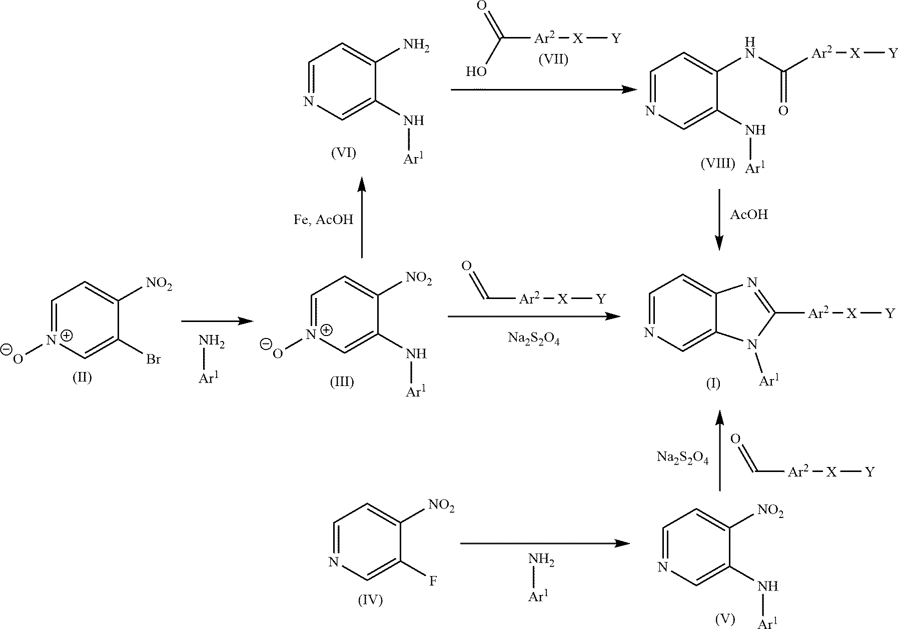

Groups Ar¹, Ar², X, and Y are as defined above.

The disclosed compounds can easily be prepared by a number of alternative routes. For example, 3-bromo-4-nitropyridine N-oxides of Formula (II) can undergo SnAr displacement with Ar¹NH₂ amines to give compounds of Formula (III), which can in turn be reductively cyclised to give compounds of Formula (I). Alternatively, 3-fluoro-4-nitropyridines of Formula (IV) can undergo SnAr displacement with Ar¹NH₂ amines to give compounds of Formula (V), which can in turn be reductively cyclised to give compounds of Formula (I). Alternatively, compounds of Formula (III) can be reduced to pyridine-3,4-diamines of Formula (VI). Compounds of Formula (VI) can then undergo amide formation with carboxylic acids of Formula (VII) to give amides of Formula (VIII) which can be cyclised to give compounds of Formula (I).

Optionally, the group Ar¹—X—Y can be built up sequentially using standard chemistry methodologies including amide, urea and sulphonamide formation. If required, standard protecting group strategies can be employed to facilitate the synthesis.

Optionally, a compound of Formula (I) can also be transformed into another compound of Formula (I) in one or more synthetic steps.

### D. Treating or Preventing Pulmonary Inflammation

In one aspect, disclosed are methods of treating or preventing pulmonary inflammation in a subject in need thereof, the method comprising the step of administering to the subject an effective amount of at least one disclosed compound, or a pharmaceutically acceptable salt thereof.

Thus, in one aspect, disclosed is a method for treating or preventing pulmonary inflammation in a subject, the method comprising administering to the subject a compound of Formula (I), or a pharmaceutically acceptable salt, solvate, hydrate, N-oxide, and/or prodrug thereof:

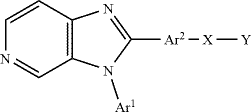

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo or C1-C4 alkyl groups; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

In one aspect, disclosed is a method for treating or preventing pulmonary inflammation in a subject, the method comprising administering to the subject a compound of Formula (I), or a pharmaceutically acceptable salt, solvate, hydrate, N-oxide, and/or prodrug thereof:

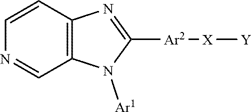

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

In one aspect, disclosed are methods of treating or preventing pulmonary inflammation in a subject in need thereof, the method comprising administering to the subject a compound having a structure represented by a formula:

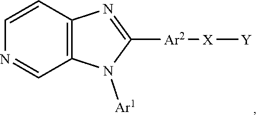

wherein Ar¹ is phenyl or a 5- or 6-membered heteroaryl, and is substituted with 0, 1, 2, or 3 halogen groups; wherein Ar² is phenyl or a 5- or 6-membered heteroaryl, and is substituted with 0, 1, 2, or 3 halogen groups; wherein X is a 3- to 7-membered heterocycloalkyl or a 3- to 7-membered cycloalkyl, and is substituted with 0, 1, 2, or 3 groups selected from halogen and C₁-C₄ alkyl; wherein Y is selected from the group consisting of 3 groups alkyl, 3 groups haloalkyl, —CN, —OR¹, —C(O)R¹, —NR²R³, —NR¹C(O)R⁴, —C(O)NR²R³, —S(O)R¹, —SO₂R¹, —S(O)NR²R³, -and SO₂NR²R³; wherein R¹ is selected from the group consisting of H, C1-C4 alkyl, and C1-C4 haloalkyl; wherein each of R² and R³ are independently selected from the group consisting of H, C1-C4 alkyl, and C1-C4 haloalkyl, or wherein each of R² and R³ together with the nitrogen to which they are attached comprise a 3- to 7-membered heterocycloalkyl substituted with 0, 1, 2, or 3 halogen groups; and wherein R⁴ is selected from the group consisting of H, C1-C4 alkyl, and C1-C4 haloalkyl, or a pharmaceutically acceptable salt thereof.

In a further aspect, the subject has been diagnosed with a need for treatment of pulmonary inflammation prior to the administering step. In a still further aspect, the subject has been diagnosed as having ALI and/or ARDS. In yet a further aspect, the subject has been diagnosed with a need for treatment of ALI and/or ARDS.

In a further aspect, the subject is a mammal. In a still further aspect, the mammal is a human.

In a further aspect, the method further comprises the step of identifying a subject in need of treatment of pulmonary inflammation.

In a further aspect, the pulmonary inflammation is caused by cytokine surge (e.g., viral induced cytokine surge) or a coronavirus (e.g., severe acute respiratory syndrome coronavirus, severe acute respiratory syndrome coronavirus 2, Middle East respiratory syndrome coronavirus).

In a further aspect, the pulmonary inflammation is caused by cytokine surge. In a still further aspect, the pulmonary inflammation is caused by viral induced cytokine surge. In yet a further aspect, the viral induced cytokine surge is associated with inflammatory macrophage-monocyte and neutrophil infiltration.

In a further aspect, the pulmonary inflammation is caused by a coronavirus. In a still further aspect, the coronavirus is SARS-CoV. In yet a further aspect, the coronavirus is SARS-CoV-2. In an even further aspect, the coronavirus is MERS-CoV.

In a further aspect, the effective amount is a therapeutically effective amount. In a still further aspect, the effective amount is a prophylactically effective amount.

In a further aspect, the method further comprises the step of administering a therapeutically effective amount of at least one agent associated with the treatment of a pulmonary inflammation. Examples of agents associated with the treatment of pulmonary inflammation include, but are not limited to, anti-inflammatories such as, for example, inhaled corticosteroids (e.g., beclomethasone dipropionate, futicasione propionate, flunisolide, budesonide, mometasone, ciclesonide, fluticasone furoate) and oral steroids (e.g., methylprednisolone, prednisolone, prednisone, hydrocortisone, dexamethasone).

In a further aspect, the compound and the agent are administered sequentially. In a still further aspect, the compound and the agent are administered simultaneously.

In a further aspect, the compound and the agent are co-formulated. In a still further aspect, the compound and the agent are co-packaged.

### E. Additional Methods of Using the Compounds

The compounds and pharmaceutical compositions of the invention are useful in treating or preventing pulmonary inflammation, such as, for example, pulmonary inflammation by cytokine surge (e.g., viral induced cytokine surge) or a coronavirus (e.g., severe acute respiratory syndrome coronavirus, severe acute respiratory syndrome coronavirus 2, Middle East respiratory syndrome coronavirus).

To treat or control the pulmonary inflammation, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of pulmonary inflammation.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of pulmonary inflammation.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Use of Compounds

In one aspect, the invention relates to the use of a disclosed compound or a product of a disclosed method. In a further aspect, a use relates to the manufacture of a medicament for the treatment of pulmonary inflammation in a subject.

Thus, in one aspect, disclosed is a compound of Formula (I), or a pharmaceutically acceptable salt, solvate, hydrate, N-oxide, and/or prodrug thereof, for use in the treatment or prevention of pulmonary inflammation:

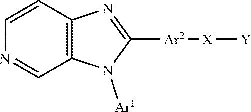

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo or C1-C4 alkyl groups; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

In one aspect, disclosed is a compound of Formula (I), or a pharmaceutically acceptable salt, solvate, hydrate, N-oxide, and/or prodrug thereof, for use in the treatment or prevention of pulmonary inflammation:

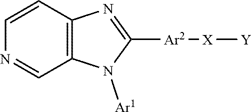

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of pulmonary inflammation in a subject. In one aspect, the use is characterized in that the subject is a human. In one aspect, the use is characterized in that the pulmonary inflammation is by cytokine surge (e.g., viral induced cytokine surge) or a coronavirus (e.g., severe acute respiratory syndrome coronavirus, severe acute respiratory syndrome coronavirus 2, Middle East respiratory syndrome coronavirus).

In a further aspect, the use relates to the manufacture of a medicament for the treatment of pulmonary inflammation in a subject.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of pulmonary inflammation in a mammal. In a further aspect, the pulmonary inflammation is by cytokine surge (e.g., viral induced cytokine surge) or a coronavirus (e.g., severe acute respiratory syndrome coronavirus, severe acute respiratory syndrome coronavirus 2, Middle East respiratory syndrome coronavirus).

### 2. Manufacture of a Medicament

In one aspect, the invention relates to a method for the manufacture of a medicament for treating pulmonary inflammation in a subject having the disorder, the method comprising combining a therapeutically effective amount of a disclosed compound or product of a disclosed method with a pharmaceutically acceptable carrier or diluent.

Thus, in one aspect, disclosed is a use of a compound of Formula (I), or a pharmaceutically acceptable salt, solvate, hydrate, N-oxide, and/or prodrug thereof, in the manufacture of a medicament for use in the treatment or prevention of pulmonary inflammation:

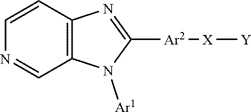

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo or C1-C4 alkyl groups; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and CC₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

In one aspect, disclosed is a use of a compound of Formula (I), or a pharmaceutically acceptable salt, solvate, hydrate, N-oxide, and/or prodrug thereof, in the manufacture of a medicament for use in the treatment or prevention of pulmonary inflammation:

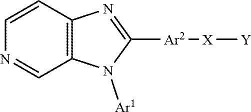

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo; X is selected from the group consisting of 3 to 7-membered heterocyclic ring and 3 to 7-membered cycloalkyl ring, each of which is optionally substituted with one or more halo; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo.

As regards these applications, the present method includes the administration to an animal, particularly a mammal, and more particularly a human, of a therapeutically effective amount of the compound effective in the treatment of pulmonary inflammation. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 3. Kits

In one aspect, disclosed are kits comprising an effective amount of a disclosed compound, and one or more of: (a) at least one agent associated with the treatment of pulmonary inflammation; (b) instructions for administering the compound in connection with treating pulmonary inflammation; and (c) instructions for treating pulmonary inflammation.

Thus, in one aspect, disclosed are kits comprising a compound of Formula (I), or a pharmaceutically acceptable salt, solvate, hydrate, N-oxide, and/or prodrug thereof:

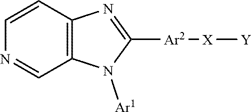

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo or C1-C4 alkyl groups; X is selected from the group consisting of 3 to 7-membered heterocyclic ring and 3 to 7-membered cycloalkyl ring, each of which is optionally substituted with one or more halo or C1-C4 alkyl groups; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo, and one or more of: (a) at least one agent associated with the treatment of pulmonary inflammation; (b) instructions for administering the compound in connection with treating pulmonary inflammation; and (c) instructions for treating pulmonary inflammation.

In one aspect, disclosed are kits comprising a compound of Formula (I), or a pharmaceutically acceptable salt, solvate, hydrate, N-oxide, and/or prodrug thereof:

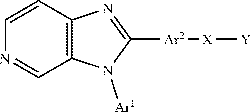

wherein Ar¹ is selected from the group consisting of phenyl and heteroaryl, each of which is optionally substituted with one or more halo; Ar² is selected from the group consisting of phenyl and heteroaryl, each of which is optionally substituted with one or more halo or C1-C4 alkyl groups; X is selected from the group consisting of 3 to 7-membered heterocyclic ring and 3 to 7-membered cycloalkyl ring, each of which is optionally substituted with one or more halo; Y is selected from the group consisting of C₁—C₄ alkyl, —CN, —OR¹, —C(O)R¹, —NR²R³, —NR¹C(O)R⁴, —C(O)NR²R³, —S(O)R¹, —SO₂R¹, —S(O)NR²R³, —SO₂NR²R³, wherein the C₁—C₄ alkyl is optionally substituted with one or more halo; R¹ is selected from the group consisting of H and C₁—C₄ alkyl, wherein the C₁—C₄ alkyl is optionally substituted with one or more halo; R² and R³ are each independently selected from the group consisting of H and C₁—C₄ alkyl, wherein the C₁—C₄ alkyl is optionally substituted with one or more halo, or R² and R³ are taken together with the nitrogen to which they are attached to form a 3 to 7-membered heterocyclic ring optionally substituted with one or more halo; and R⁴ is selected from the group consisting of H and C₁—C₄ alkyl, wherein the C₁—C₄ alkyl is optionally substituted with one or more halo, and one or more of: (a) at least one agent associated with the treatment of pulmonary inflammation; (b) instructions for administering the compound in connection with treating pulmonary inflammation; and (c) instructions for treating pulmonary inflammation.

In one aspect, disclosed are kits comprising a compound having a structure represented by a formula:

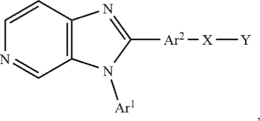

wherein Ar¹ is phenyl or a 5- or 6-membered heteroaryl, and is substituted with 0, 1, 2, or 3 halogen groups; wherein Ar² is phenyl or a 5- or 6-membered heteroaryl, and is substituted with 0, 1, 2, or 3 halogen groups; wherein X is a 3- to 7-membered heterocycloalkyl or a 3- to 7-membered cycloalkyl, and is substituted with 0, 1, 2, or 3 groups selected from halogen and C₁—C₄ alkyl; wherein Y is selected from the group consisting of C1-C4 alkyl, C1-C4 haloalkyl, —CN, —OR¹, —C(O)R¹, —NR²R³, —NR¹C(O)R⁴, —C(O)NR²R³, —S(O)R¹, —SO₂R¹, —S(O)NR²R³, -and SO₂NR²R³; wherein R¹ is selected from the group consisting of H, C1-C4 alkyl, and C1-C4 haloalkyl; wherein each of R² and R³ are independently selected from the group consisting of H, C1-C4 alkyl, and C1-C4 haloalkyl, or wherein each of R² and R³ together with the nitrogen to which they are attached comprise a 3- to 7-membered heterocycloalkyl substituted with 0, 1, 2, or 3 halogen groups; and wherein R⁴ is selected from the group consisting of H, C1-C4 alkyl, and C1-C4 haloalkyl, or a pharmaceutically acceptable salt thereof, and one or more of: (a) at least one agent associated with the treatment of pulmonary inflammation; (b) instructions for administering the compound in connection with treating pulmonary inflammation; and (c) instructions for treating pulmonary inflammation.

In a further aspect, the pulmonary inflammation is by cytokine surge (e.g., viral induced cytokine surge) or a coronavirus (e.g., severe acute respiratory syndrome coronavirus, severe acute respiratory syndrome coronavirus 2, Middle East respiratory syndrome coronavirus).

In a further aspect, the agent is selected from anti-inflammatories such as, for example, inhaled corticosteroids (e.g., beclomethasone dipropionate, futicasione propionate, flunisolide, budesonide, mometasone, ciclesonide, fluticasone furoate) and oral steroids (e.g., methylprednisolone, prednisolone, prednisone, hydrocortisone, dexamethasone).

In a further aspect, the compound and the agent are co-formulated. In a further aspect, the compound and the agent are co-packaged.

In a further aspect, the compound and the agent are administered sequentially. In a still further aspect, the compound and the agent are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

The above-mentioned compounds, compositions, methods, compounds for use, and uses are the compounds, compositions, methods, compounds for use, and uses of the invention. For brevity, they may be collectively referred to as “methods of the invention.”

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### F. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

The following abbreviations have been used:

| | |
|---|---|
| Ac | acetyl |
| aq | aqueous |
| Boc | tertiary-butyloxycarbonyl |
| calcd | calculated |
| d | day(s) |
| DCM | Dichloromethane |
| DIPEA | Diisopropylethylamine |
| DMA | dimethylacetamide |
| DMF | dimethylformamide |
| DMSO | Dimethyl sulfoxide |
| EDC | N-(3-dimethylaminopropyl)-N′-ethylcarbodiimide |
| ES⁺ | electrospray ionization |
| Et₃N | Trimethylamine |
| EtOAc | ethyl acetate |
| EtOH | ethanol |
| Ex | Example |
| h | hour(s) |
| HATU | O-(7-azabenzotriazol-1-y1)-N,N,N·,N·-tetramethyluronium hexafluorophosphate |
| HBTU | O-benzotriazole-N,N,N′,N′-tetramethyl-uronium-hexafluoro phosphate |
| HPLC | High Performance Liquid Chromatography |
| Int | Intermediate |
| LCMS | Liquid Chromatography Mass Spectrometry |
| LDA | Lithium diisopropylamide |
| M | Molar |
| MeCN | Acetonitrile |
| MeOH | Methanol |
| [MH]⁺ | protonated molecular ion |
| Min | minute(s) |
| NMP | 1-methyl-2-pyrrolidinone |
| QTOF | Quadrupole time-of-flight mass spectrometer |
| RP | reverse phase |
| RT | room temperature |
| Rt | retention time |
| sat | saturated |
| TFA | trifluoroacetic acid |
| THF | Tetrahydrofuran |
| UV | Ultra violet |
| XPhos | 2-dicyclohexylphosphino-2′,4′,6′-triisopropylbiphenyl |

### 1. Chemistry Experimental Methods

Reactions were conducted at room temperature unless otherwise specified. Microwave reactions were performed with a Biotage microwave reactor using process vials fitted with aluminium caps and septa. Preparative chromatography was performed using a Flash Master Personal system equipped with Isolute Flash II silica columns or using a CombiFlash Companion system equipped with GraceResolv silica column. Reverse Phase HPLC was performed on a Gilson system with a UV detector equipped with Phenomenex Synergi Hydro RP 150×10 mm, or YMC ODS-A 100/150×20 mm columns. The purest fractions were collected, concentrated and dried under vacuum. Compounds were typically dried in a vacuum oven at 40° C. prior to purity analysis. Compound analysis was performed by HPLC/LCMS using an Agilent 1100 HPLC system / Waters ZQ mass spectrometer connected to an Agilent 1100 HPLC system with a Phenomenex Synergi, RP-Hydro column (150×4.6 mm, 4 µm, 1.5 mL per min, 30° C., gradient 5-100% MeCN (⁺0.085% TFA) in water (⁺0.1% TFA) over 7 min, 200-300 nm). Accurate masses were measured using a Waters QTOF electrospray ion source and corrected using Leucine Enkephalin lockmass. Spectra were acquired in positive and negative electrospray mode. The acquired mass range was m/z 100-1000. Test compounds were dissolved in DMSO to give a 10 mM stock solution. Typically 5 mL of the DMSO stock were diluted with 495 mL of acetonitrile and then further diluted with acetonitrile and water (1:1) to give a final concentration of 0.2 mM. The mass values reported correspond either to the parent molecule with a hydrogen added [MH] or with a hydrogen subtracted [M-H]. The compounds prepared were named using IUPAC nomenclature.

The disclosed compounds may be synthesized as set out in WO 2014/140592 (the contents of which is incorporated herein).

### 1. Intermediate 1: 3-[(4-Chlorophenyl)Amino]-4-Nitropyridin-1-Ium-1-Olate

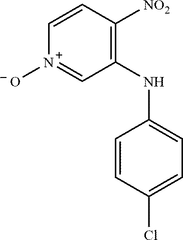 [SMILES: O=[N+]([O-])c1cc[n+]([O-])cc1Nc1ccc(Cl)cc1]

3-Bromo-4-nitropyridine N-oxide (1.00 g, 4.57 mmol) and 4-chloroaniline (1.75 g, 13.7 mmol) were dissolved in EtOH and heated at 60° C. for 18 h. The reaction mixture was cooled to 0° C. and the precipitate was collected by filtration and washed with cold EtOH to give the title compound as an orange solid (317 mg, 26.1%). LCMS (ES+): 266.1 [MH]+. HPLC: Rt 5.44 min, 99.5% purity.

### M. Intermediate 2

Intermediate 2 was prepared similarly to Intermediate 1, by coupling of 3-bromo-4-nitropyridine N-oxide with the appropriate aniline; see Table 1 below. TABLE 1: SNAR FORMATION OF ANILINES

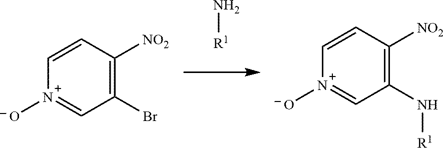

| Int | Structure | Name | Form, Yield, LCMS, HPLC |
|---|---|---|---|
| 2 | 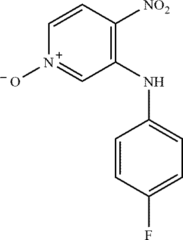 [SMILES: O=[N+]([O-])c1cc[n+]([O-])cc1Nc1ccc(F)cc1] | 3-[(4-Fluorophenyl)amino] -4-nitropyridin-1-ium-1-olate | Orange solid Yield 2.66 g, 46.7% LCMS (ES⁺): 250.1 [MH]⁺ HPLC: Rt 5.00 min, 97.3% purity |

### N. Intermediate 3: 3-N-(4-Chlorophenyl)Pyridine-3,4-Diamine

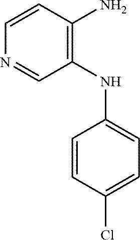 [SMILES: Nc1ccncc1Nc1ccc(Cl)cc1]

Intermediate 1 (317 mg, 1.19 mmol) was dissolved in AcOH (10 mL) and iron powder (333 mg, 5.97 mmol) was added. The reaction mixture was heated at reflux for 1 h, diluted with water (50 mL), basified with Na₂CO₃ and extracted into DCM (3×50 mL). The combined organic fractions were dried (MgSO₄) and concentrated in vacuo to give the title compound as a red gum (254 mg, 96.9%). LCMS (ES⁺): 220.2 [MH]⁺. HPLC: Rt 4.31 min, 99.5% purity.

### O. Intermediate 4: 6-(2-Methylmorpholin-4-YL)Pyridine-3-Carbaldehyde

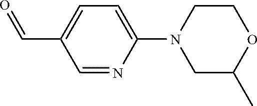

2-Chloro-5-pyridinecarboxaldehyde (500 mg, 3.53 mmol) and 2-methylmorpholine (750 mg, 7.42 mmol) were dissolved in DMF (2 mL) and the reaction mixture was heated at 100° C. in a microwave reactor for 20 min and concentrated in vacuo. The residue was suspended in dioxane (5 mL), filtered and concentrated in vacuo to give the title compound (730 mg, 100%) as an orange gum. LCMS (ES⁺): 207.1 [MH]⁺.

### P. Intermediate 5: Tert-Butyl N-{1-[5-({3-[(4-Chlorophenyl)Amino]Pyridin-4-YL}Carbamoyl)Pyridin-2-Yl]Piperidin-4-YL}Carbamate

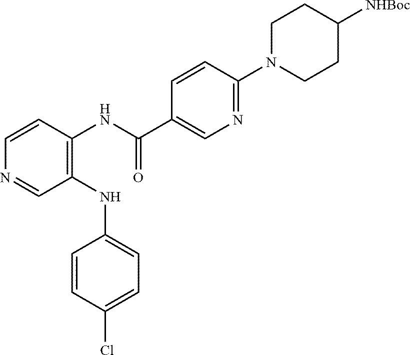 [SMILES: CC(C)(C)OC(=O)NC1CCN(c2ccc(C(=O)Nc3ccncc3Nc3ccc(Cl)cc3)cn2)CC1]

Intermediate 3, Intermediate 16 and DIPEA were dissolved in DMF and EDC was added. The reaction mixture was stirred for 18 h and further Intermediate 16 and EDC were added. The reaction mixture was stirred for 5 h, diluted with 1 M aq. Na₂CO₃ and extracted into DCM. The combined organic fractions were dried (MgSO₄) and concentrated in vacuo. The residue was purified by column chromatography to give the title compound as an off white solid (954 mg, 76.4%). LCMS (ES⁺): 523.1 [MH]⁺ HPLC: Rt 5.16 min, 97.8% purity.

### Q. Intermediate 6: 1-{5-[3-(4-Fluorophenyl)-3H-Imidazo[4,5-C]Pyridin-2-Yl]Pyridin-2-Yl}Piperazine Trihydrochloride

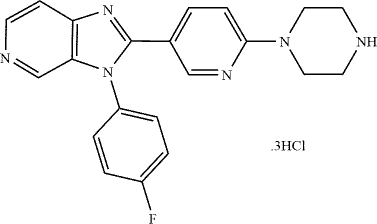

Intermediate 6 was prepared similarly to Intermediate 14, using Intermediate 2 instead of Intermediate 1, to give the title compound (684 mg, 100%) as a white solid. LCMS (ES⁺): 375.1 [MH]⁺.

### R. Intermediate 7: 2-Chloro-5-[3-(4-Fluorophenyl)-3H-Imidazo[4,5-C]Pyridin-2-Yl]Pyridine

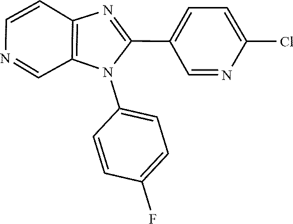 [SMILES: Fc1ccc(-n2c(-c3ccc(Cl)nc3)nc3ccncc32)cc1]

Intermediate 2 (1.00 g, 4.01 mmol) and 2-chloro-5-pyridinecarboxaldehyde (682 mg, 4.82 mmol) were dissolved in EtOH (8 mL) and Na₂S₂O₄ (2.79 g, 16.1 mmol) was added. The reaction mixture was heated in a microwave reactor at 160° C. for 1 h, diluted with water (25 mL) and NaHCO₃ (25 mL) and extracted into DCM (3×50 mL). The combined organic fractions were dried (MgSO₄) and concentrated in vacuo. The residue was purified by column chromatography to give the title compound (375 mg, 28.8%) as a yellow oil. LCMS (ES⁺): 325.1 [MH]⁺.

### S. Intermediate 8: 1-{5-[3-(4-Fluorophenyl)-3H-Imidazo[4,5-C]Pyridin-2-Yl]Pyridin-2-Yl}-1,4-Diazepane

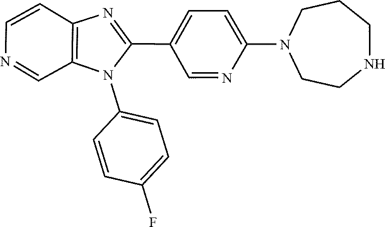 [SMILES: Fc1ccc(-n2c(-c3ccc(N4CCCNCC4)nc3)nc3ccncc32)cc1]

Intermediate 7 (375 mg, 1.15 mmol) and homopiperazine (578 mg, 5.77 mmol) were dissolved in DMA (6 mL) and the reaction mixture was heated in a microwave reactor at 180° C. for 30 min and concentrated in vacuo. The residue was partitioned between DCM (50 mL) and sat. aq. Na₂CO₃ (50 mL) and the organic fraction dried (MgSO₄) and concentrated in vacuo to give the title compound (410 mg, 91.5%) as a red oil. LCMS (ES⁺): 389.2 [MH]⁺.

### T. Intermediate 9: 3-N-(4-Chlorophenyl)Pyridine-3,4-Diamine

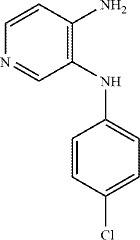 [SMILES: Nc1ccncc1Nc1ccc(Cl)cc1]

Intermediate 1 (317 mg, 1.19 mmol) was dissolved in AcOH (10 mL) and iron powder (333 mg, 5.97 mmol) was added. The reaction mixture was heated at reflux for 1 h, diluted with water (50 mL), basified with Na₂CO₃ and extracted into DCM (3×50 mL). The combined organic fractions were dried (MgSO₄) and concentrated in vacuo to give the title compound as a red gum (254 mg, 96.9%). LCMS (ES⁺): 220.2 [MH]⁺. HPLC: Rt 4.31 min, 99.5% purity.

### U. Intermediate 10: Tert-Butyl 4-[5-(Methoxycarbonyl)-1,3-Oxazol-2-Yl]Piperazine-1-Carboxylate

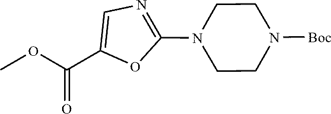 [SMILES: COC(=O)c1cnc(N2CCN(C(=O)OC(C)(C)C)CC2)o1]

Methyl 5-chloro-2-oxazolcarboxylate, Et3N and tert-butyl piperazine-1-carboxylate were dissolved in dioxane and heated in a microwave reactor at 100° C. for 20 min. Water (50 mL) and brine (25 mL) were added and the reaction mixture was extracted into EtOAc (2 × 100 mL), dried (MgSO₄) and concentrated in vacuo to give the title compound as a pale yellow solid (406 mg, 42.1%). LCMS (ES⁺): 334.2 [MNa]⁺. HPLC: Rt 5.81 min, 97.1% purity.

### V. Intermediate 11: 2-{4-[(Tert-Butoxy)Carbonyl] Piperazin-1-Yl}-1,3-Oxazole-5-Carboxylic Acid

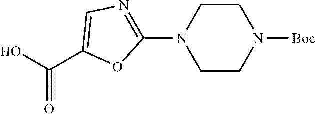 [SMILES: CC(C)(C)OC(=O)N1CCN(c2ncc(C(=O)O)o2)CC1]

Intermediate 10 was dissolved in THF/water (1:1), lithium hydroxide monohydrate was added and the reaction mixture was stirred for 20 min. 1 M aq. HCl was added and the reaction mixture was extracted with EtOAc, dried (MgSO₄) and concentrated in vacuo to give the title compound as a white solid (324 mg, 84.4%). LCMS (ES⁺): 320.1 [MNa]⁺ HPLC: Rt 4.77 min, 100% purity.

### W. Intermediate 12: Tert-Butyl 4-[5-({3-[(4-Chlorophenyl)Amino]Pyridin-4-Yl}Carbamoyl)-1,3-Oxazol-2-Yl]Piperazine-1-Carboxylate

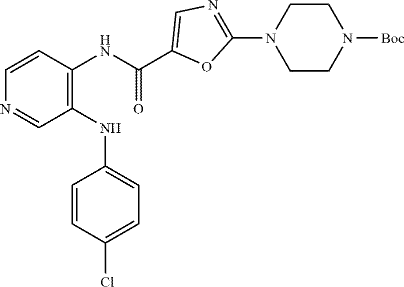 [SMILES: CC(C)(C)OC(=O)N1CCN(c2ncc(C(=O)Nc3ccncc3Nc3ccc(Cl)cc3)o2)CC1]

Intermediate 9, intermediate 11 and DIPEA were dissolved in DMF and EDC was added. The reaction mixture was stirred for 18 h and further pyridine-3-carboxylic acid and EDC were added. The reaction mixture was stirred for 5 h, diluted with 1 M aq. Na₂CO₃ and extracted into DCM. The combined organic fractions were dried (MgSO₄) and concentrated in vacuo. The residue was purified by column chromatography to give the title compound as an orange solid. Used crude (602 mg). LCMS (ES⁺): 499.0 [MH]⁺. HPLC: Rt 5.76 min, 76.4% purity.

### X. Intermediate 13: 1-{5-[3-(4-Chlorophenyl)-3H-Imidazo[4,5-C]Pyridin-2-Yl]-1,3-Oxazol-2-Yl}Piperazine Dihydrochloride

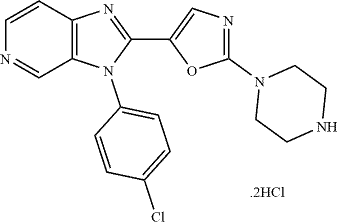

Intermediate 12 was dissolved in AcOH and heated using a microwave reactor at 100° C. for 15 min, diluted with water, basified with Na₂CO₃ and extracted into DCM. The combined organic fractions were dried (MgSO₄) and concentrated in vacuo. The residue was purified by column chromatography to give the title compound as an orange solid (58.0 mg, 11.8%; additional Boc deprotection step included). HRMS (ESI⁺) calcd for [MH]⁺ of C₁₉H₁₇ClN₆O 381.1230 found 381.1241. HPLC: Rt 3.25 min, 100% purity.

### Y. Intermediate 14: 1-{5-[3-(4-Chlorophenyl)-3H-Imidazo[4,5-C]Pyridin-2-Yl]Pyridin-2-Yl}Piperazine

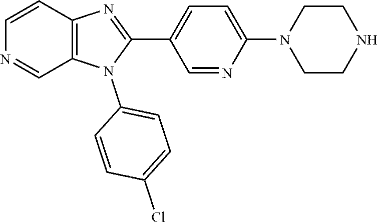 [SMILES: Clc1ccc(-n2c(-c3ccc(N4CCNCC4)nc3)nc3ccncc32)cc1]

Intermediate 1 and 2-{4-[(tert-butoxy)carbonyl]piperazin-1-yl}-pyridin-5-carboxylic acid were dissolved in EtOH and Na₂S₂O₄ was added. The reaction mixture was heated using a microwave reactor at 160° C. for 1 h, diluted with sat. aq. NaHCO₃ and water, and extracted into DCM. The combined organic fractions were dried (MgSO₄) and concentrated in vacuo. The residue was purified by column chromatography to give the title compound as a yellow solid (3.10 mg, 1.39%; additional Boc deprotection step included). HRMS (ESI⁺) calcd for [MH]⁺ of C₂₁H₁₉ClN₆ 391.1438 found 391.1427. HPLC: Rt 3.51 min, 100% purity.

### Z. Intermediate 15: Methyl 6-(4-{[(Tert-Butzoxy)Carbonyl]Amino}Piperidin-1-Yl)Pyridine-3-Carboxylate

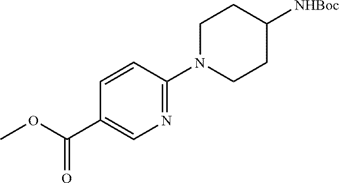 [SMILES: COC(=O)c1ccc(N2CCC(NC(=O)OC(C)(C)C)CC2)nc1]

Methyl 2-chloro-5-pyridinecarboxylate, Et₃N and 4-N-(tert-butylcarbamate)piperidine were dissolved in dioxane and heated in a microwave reactor at 100° C. for 20 min. Water and brine were added and the reaction mixture was extracted into EtOAc, dried (MgSO₄) and concentrated in vacuo to give the title compound as an off white solid (1.66 g, 84.9%). LCMS (ES⁺): 336.1 [MH]⁺ HPLC: Rt 4.73 min, 98.2% purity.

### Aa. Intermediate 16: Lithium 6-(4-{[(Tert-Butoxy) Carbonyl] Amino}Piperidin-1-Yl)Pyridine-3-Carboxylate

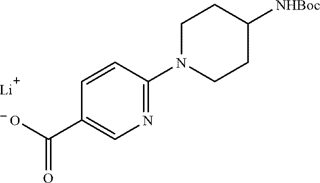 [SMILES: [Li+] ; CC(C)(C)OC(=O)NC1CCN(c2ccc(C(=O)[O-])cn2)CC1]

Intermediate 15 (was dissolved in THF/water (1:1), lithium hydroxide monohydrate was added and the reaction mixture was stirred for 20 min. 1 M aq. HCl (5 mL) was added and the reaction mixture was extracted with EtOAc (2× 100 mL), dried (MgSO₄) and concentrated in vacuo to give the title compound as a white solid (used crude). LCMS (ES⁺): 322.1 [MH]⁺ HPLC: Rt 4.20 min, 96.8% purity.

### Bb. Example 1: 1-{5-[3-(4-Chlorophenyl)-3H-Imidazo[4,5-C]Pyridin-2-Yl]Pyridin-2-Yl}Piperidin-4-Amine

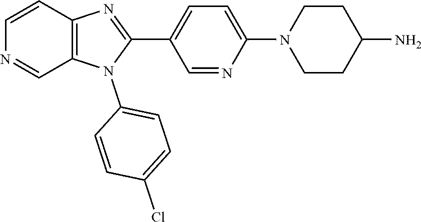 [SMILES: NC1CCN(c2ccc(-c3nc4ccncc4n3-c3ccc(Cl)cc3)cn2)CC1]

Intermediate 1 was dissolved in AcOH and heated using a microwave reactor at 100° C. for 15 min, diluted with water, basified with Na₂CO₃ and extracted into DCM. The combined organic fractions were dried (MgSO₄) and concentrated in vacuo. The residue was purified by column chromatography to give the title compound as a white solid (24.2 mg, 38.7%). HRMS (ESI⁺) calcd for [MH]⁺ of C₂₂H₂₁ClN₆ 405.1594 found 405.1591. HPLC: Rt 3.52 min, 100% purity.

### Cc. Example 2: 4-{5-[3-(4-Fluorophenyl)-3H-Imidazo[4,5-C]Pyridin-2-Yl]Pyridin-2-Yl}-2-Methylmorpholine

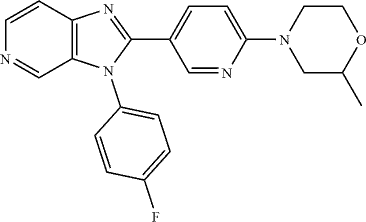 [SMILES: CC1CN(c2ccc(-c3nc4ccncc4n3-c3ccc(F)cc3)cn2)CCO1]

Intermediate 2 and intermediate 4 were dissolved in EtOH and Na₂S₂O₄ was added. The reaction mixture was heated using a microwave reactor at 160° C. for 1 h, diluted with sat. aq. NaHCO₃ and water, and extracted into DCM. The combined organic fractions were dried (MgSO₄) and concentrated in vacuo. The residue was purified by column chromatography to give the title compound as an orange solid (42.7 mg, 9.11%). HRMS (ESI⁺) calcd for [MH]⁺ of C₂₂H₂₀FN₅O 390.1730 found 390.1726. HPLC: Rt 4.54 min, 99.4% purity.

### Dd. Example 3: N-(1-{5-[3-(4-Chlorophenyl)-3H-Imidazo[4,5-C]Pyridin-2-Yl]Pyridin-2-YL}Piperidin-4-Yl)Acetamide

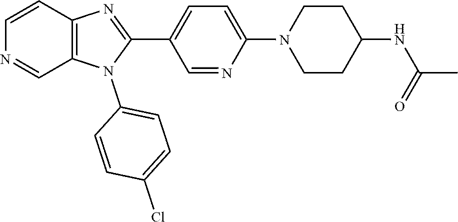 [SMILES: CC(=O)NC1CCN(c2ccc(-c3nc4ccncc4n3-c3ccc(Cl)cc3)cn2)CC1]

Example 1 (100 mg, 0.247 mmol), Et₃N (41.2 µL, 0.296 mmol) and acetyl chloride (19.3 µL, 0.272 mmol) were dissolved in DCM (10 mL) and the reaction mixture was stirred for 2 h and concentrated in vacuo. The residue was purified by column chromatography and partitioned between DCM (20 mL) and sat. aq. NaHCO₃ (20 mL). The organic fraction was washed with sat. aq. NaHCO₃ (20 mL), dried (MgSO₄) and concentrated in vacuo to give the title compound (61.6 mg, 55.8%) as a light yellow solid. HRMS (ESI⁺) calcd for [MH]⁺ of C₂₄H₂₃ClN₆O 447.1700 found 447.1701. HPLC: Rt 3.98 min, 99.7% purity.

### Ee. Example 4: 1-(4-{5-[3-(4-Fluorophenyl)-3H-Imidazo[4,5-C]Pyridin-2-Yl]Pyridin-2-Yl}Piperazin-1-YL)Ethan-1-One

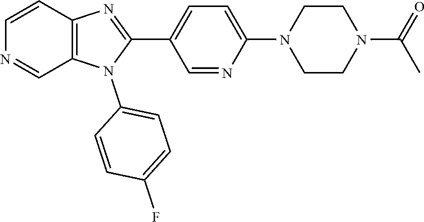 [SMILES: CC(=O)N1CCN(c2ccc(-c3nc4ccncc4n3-c3ccc(F)cc3)cn2)CC1]

Example 4 was prepared similarly to Example 3, using Intermediate 6 instead of Example 1, to give the title compound (227 mg, 38.7%) as a white solid. HRMS (ESI⁺) calcd for [MH]⁺ of C₂₃H₂₁FN₆O 417.1839 found 417.1851. HPLC: Rt 4.26 min, 100% purity.

### Ff. Example 5: 1-(4-{5-[3-(4-Fluorophenyl)-3H-Imidazo[4,5-C]Pyridin-2-Yl]Pyridin-2-YL}-1,4-Diazepan-1-Yl)Ethan-1-One; Bis(Trifluoroacetic Acid)

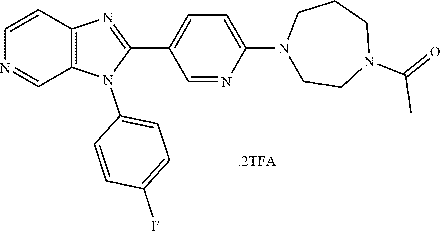

Example 5 was prepared similarly to Example 3, using Intermediate 8 instead of Example 1, to give the title compound (143 mg, 41.2%) as a pink gum. HRMS (ESI⁺) calcd for [MH]⁺ of C₂₄H₂₃FN₆O 431.1996 found 431.1997. HPLC: Rt 4.41 min, 99.7% purity.

### Gg. Example 6: N-(1-{5-[3-(4-Chlorophenyl)-3H-Imidazo[4,5-C]Pyridin-2-Yl]Pyridin-2-Yl}Piperidin-4-YL)Methanesulfonamide

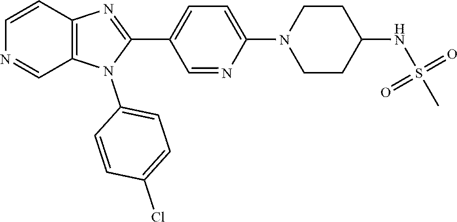 [SMILES: CS(=O)(=O)NC1CCN(c2ccc(-c3nc4ccncc4n3-c3ccc(Cl)cc3)cn2)CC1]

Example 1 (100 mg, 0.247 mmol), Et₃N (41.2 µL, 0.296 mmol) and methanesulfonyl chloride (26.8 µL, 0.346 mmol) were dissolved in DCM (10 mL) and the reaction mixture was stirred for 2 h, diluted with DCM (20 mL), washed with sat. aq. NaHCO₃ (30 mL), dried (MgSO₄) and concentrated in vacuo. The residue was triturated from MeOH (3 mL) and collected by filtration to give the title compound (30.6 mg, 25.7%) as a yellow solid. HRMS (ESI⁺) calcd for [MH]⁺ of C₂₃H₂₃ClN₆O₂S 483.1370 found 483.1375. HPLC: Rt 4.18 min, 99.4% purity.

### Hh. Example 7: 1-{5-[3-(4-Fluorophenyl)-3H-Imidazo[4,5-C]Pyridin-2-Yl]Pyridin-2-Yl}-4-Methanesulfonylpiperazine

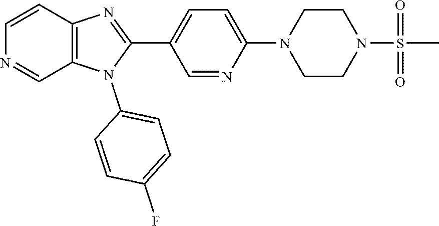 [SMILES: CS(=O)(=O)N1CCN(c2ccc(-c3nc4ccncc4n3-c3ccc(F)cc3)cn2)CC1]

Example 7 was prepared similarly to Example 6, using Intermediate 6 instead of Example 1, to give the title compound (44.5 mg, 10.3%) as a yellow solid. HRMS (ESI⁺) calcd for [MH]⁺ of C₂₂H₂₁FN₆O₂S 453.1509 found 453.1522. HPLC: Rt 4.59 min, 98.2% purity.

### Ii. Example 8: 4-{5-[3-(4-Chlorophenyl)-3H-Imidazo[4,5-C]Pyridin-2-Yl]Pyridin-2-Yl}Piperazine-1-Carboxamide Dihydrochloride

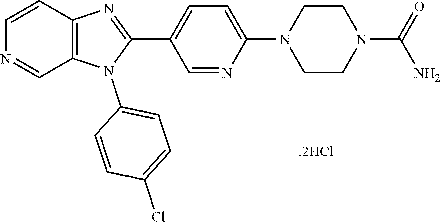

Intermediate 14 trihydrochloride (94.5 mg, 0.189 mmol) was dissolved in DCM (5 mL), and DIPEA (145 µL, 0.831 mmol) and trimethylsilyl isocyanate (30.7 µL, 0.227 mmol) were added. The reaction mixture was stirred for 16h, diluted with 1M aq. Na₂CO₃ (25 mL) and extracted into DCM (3x25 mL). The combined organic fractions were dried (MgSO₄) and concentrated in vacuo. The residue was dissolved in 1.25 M HCl in EtOH (5 mL), stirred for 1h and concentrated in vacuo. The residue was purified by reverse phase HPLC to give the title compound (21.4 mg, 22.3%) as a yellow solid. HRMS (ESI⁺) calcd for [MH]⁺ of C₂₂H₂₀ClN₇O 434.1496 found 434.1497. HPLC: Rt 4.19 min, 98.5% purity.

### Jj. Examples 9 TO 11

Examples 9-11 were prepared similarly to Example 8, by reaction of Intermediates 6, 8, and 13 with trimethylsilyl isocyanate; see Table 2 below.

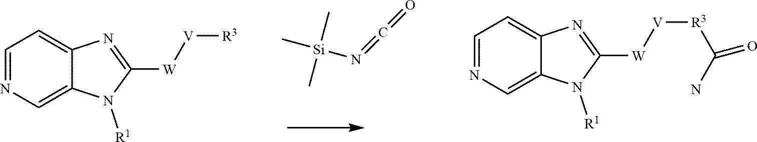

TABLE 2

REACTION OF INTERMEDIATES 6, 8 AND 13 WITH TRIMETHYLSILYL ISOCYANATE

| Ex | Structure | Name | Intermediate(s) used, Form, Yield, LCMS, HPLC |
|---|---|---|---|
| 9 | 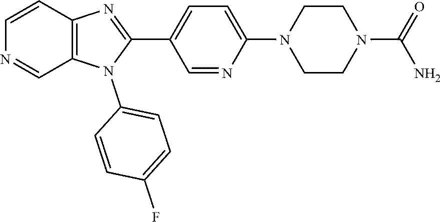 [SMILES: NC(=O)N1CCN(c2ccc(-c3nc4ccncc4n3-c3ccc(F)cc3)cn2)CC1] | 4-{5-[3-(4-Fluorophenyl)-3H-imidazo[4,5-c]pyridin-2-yl]pyridin-2-yl}piperazine-1-carboxamide | From Intermediate 6 White solid Yield 44. 0 mg, 11.1% HRMS (ESI⁺) calcd for [MH]⁺ of C₂₂H₂₀FN₇O 418.1791 found 418.1795. HPLC: Rt 3.88 min, 100% purity |
| 10 | 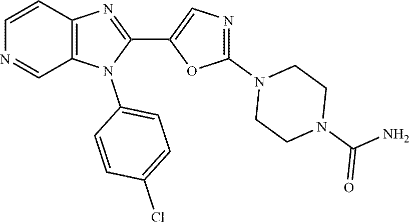 [SMILES: NC(=O)N1CCN(c2ncc(-c3nc4ccncc4n3-c3ccc(Cl)cc3)o2)CC1] | 4-{5-[3-(4-Chlorophenyl)-3H-imidazo[4,5-c]pyridin-2-yl]-1,3-oxazol-2-yl}piperazine-1-carboxamide | From Intermediate 13 Pale yellow solid Yield 7.20 mg, 7.71% HRMS (ESI⁺) calcd for [MH]⁺ of C₂₀H₁₈ClN₇O₂ 424.1289 found 424.1288. HPLC: Rt 4.20 min, 99.7% purity |
| 11 | 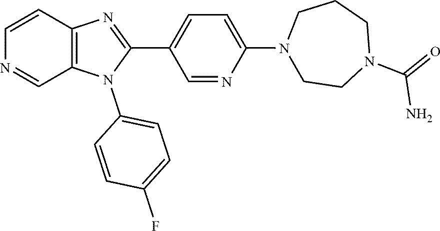 [SMILES: NC(=O)N1CCCN(c2ccc(-c3nc4ccncc4n3-c3ccc(F)cc3)cn2)CC1] | 4-{5-[3-(4-Fluorophenyl)-3H-imidazo[4,5-c]pyridin-2-yl]pyridin-2-yl}-1,4-diazepane-1-carboxamide | From Intermediate 8 Pink solid Yield 56.7 mg, 24.9% HRMS (ESI⁺) calcd for [MH]⁺ of C₂₃H₂₂FN₇O 432.1948 found 432.1955. HPLC: Rt 3.83 min, 99.0% purity |

### 2. Biological Tests

### A. Biological Assays of the SSAO Enzyme Inhibitors

All primary assays were performed at room temperature with purified recombinantly expressed human SSAO. Enzyme was prepared essentially as described in Ohman et al. (Protein Expression and Purification 46 (2006) 321-331). In addition, secondary and selectivity assays were performed using SSAO prepared from various tissues or purified rat recombinant SSAO. The enzyme activity was assayed with benzylamine as substrate by measuring either benzaldehyde production, using ¹⁴C-labeled substrate, or by utilizing the production of hydrogen peroxide in a horseradish peroxidase (HRP) coupled reaction. Test compounds were dissolved in dimethyl sulfoxide (DMSO) to a concentration of 10 mM. Dose-response measurements were assayed by either creating 1:10 serial dilutions in DMSO to produce a 7-point curve or by making 1:3 serial dilutions in DMSO to produce 11-point curves. The top concentrations were adjusted depending on the potency of the compounds and subsequent dilution in reaction buffer yielded a final DMSO concentration ≤ 2%.

### B. Hydrogen Peroxide Detection

In a horseradish peroxidase (HRP) coupled reaction, hydrogen peroxide oxidation of 10-acetyl-3,7-dihydroxyphenoxazine produced resorufin, which is a highly fluorescent compound (Zhout and Panchuk-Voloshina. Analytical Biochemistry 253 (1997) 169-174; Amplex® Red Hydrogen Peroxide/peroxidase Assay kit, Invitrogen A22188). Enzyme and compounds in 50 mM sodium phosphate, pH 7.4 were set to pre-incubate in flat-bottomed microtiter plates for approximately 15 min before initiating the reaction by addition of a mixture of HRP, benzylamine and Amplex reagent. Benzylamine concentration was fixed at a concentration corresponding to the Michaelis constant, determined using standard procedures. Fluorescence intensity was then measured at several time points during 1-2 hr, exciting at 544 nm and reading the emission at 590 nm. For the human SSAO assay final concentrations of the reagents in the assay wells were: SSAO enzyme 1 ug/ mL, benzylamine 100 µM, Amplex reagent 20 µM, HRP 0.1 U/ mL and varying concentrations of test compound. The inhibition was measured as % decrease of the signal compared to a control without inhibitor (only diluted DMSO). The background signal from a sample containing no SSAO enzyme was subtracted from all data points. Data was fitted to a four parameter logistic model and IC₅₀ values were calculated using the GraphPad Prism 4 or XLfit 4 programs.

### C. Aldehyde Detection

SSAO activity was assayed using ¹⁴C-labeled benzylamine and analysed by measuring radioactive benzaldehyde. In a white 96-well optiplate (Packard), 20 µL of diluted test compound was pre-incubated at room temperature with 20 µL SSAO enzyme for approximately 15 min with continuous agitation. All dilutions were made with PBS. The reaction was initiated by adding 20 µL of the benzylamine substrate solution containing [7-¹⁴C] Benzylamine hydrochloride (CFA589, GE Healthcare). The plate was incubated for 1 h as above after which the reaction was stopped by acidification (10 µL 1 M aq HCl). Then 90 µL Micro Scint-E solution (Perkin-Elmer) was added to each well and the plate was continuously mixed for 15 min. Phase separation occurred instantly and activity was read in a Topcount scintillation counter (Perkin-Elmer). In the final reaction well, the human recombinant SSAO concentration was 10 µg/mL. In order to optimize sensitivity, the substrate concentration was decreased as compared to the HRP coupled assay in order to get a higher fraction of radioactive product. In the human SSAO assay, benzylamine concentration was 40 µM (0.2 µCi/mL). Data was analysed as above.

All of the exemplified compounds of the invention had an IC₅₀ value of between 1 nM and 1200 nM at SSAO (see Table 3 below).

TABLE 3

SSAO INHIBITORY ACTIVITY (A: <50 NM, B: 50-200 NM, C: 200-1200 NM)

| Compound | SSAO IC₅₀ (nM) |
|---|---|
| 1 | A |
| 2 | A |
| 3 | A |
| 4 | A |
| 5 | A |
| 6 | A |
| 7 | A |
| 8 | A |
| 9 | A |
| 10 | B |
| 11 | C |

### D. Herg Assay

Compounds of the invention were tested for inhibition of the human ether a go-go related gene (hERG) K⁺ channel using IonWorks patch clamp electrophysiology. 8-Point concentration-response curves were generated on two occasions using 3-fold serial dilutions from the maximum assay concentration (11 µM). Electrophysiological recordings were made from a Chinese Hamster Lung cell line stably expressing the full length hERG channel. Single cell ion currents were measured in the perforated patch clamp configuration (100 µg/mL amphoterocin) at room temperature using an IonWorks Quattro instrument. The internal solution contained 140 mM KCl, 1 mM MgCl₂, 1 mM EGTA, and 20 mM HEPES and was buffered to pH 7.3. The external solution contained 138 mM NaCl, 2.7 mM KCl, 0.9 mM CaCl₂, 0.5 mM MgCl₂, 8 mM Na₂HPO₄, and 1.5 mM KH₂PO₄, and was buffered to pH 7.3. Cells were clamped at a holding potential of 70 mV for 30 s and then stepped to +40 mV for 1 s. This was followed by a hyperpolarising step of 1 s to 30 mV to evoke the hERG tail current. This sequence was repeated 5 times at a frequency of 0.25 Hz. Currents were measured from the tail step at the 5^{th} pulse, and referenced to the holding current. Compounds were incubated for 6-7 min prior to a second measurement of the hERG signal using an identical pulse train. A minimum of 17 cells were required for each pIC₅₀ curve fit. A control compound (quinidine) was used (see Table 4 below).

TABLE 4

HERG IC50 (A: >10 µM, B: 1-10 µM)

| Compound | hERG IC₅₀ |
|---|---|
| 2 | A |
| 3 | A |
| 4 | A |
| 6 | B |
| 7 | A |
| 9 | A |

### E. LPS-Induced Pulmonary Inflammation Study

This study was to test the efficacy of orally administered compounds as disclosed herein to reduce LPS-induced inflammatory cell infiltration of the lungs, and was carried out in male C57BI/6 mice.

Day 1: 70 C57BI/6 mice (Envigo, male, 6-8 weeks old) were received, individually examined and housed in seven cages of ten mice each. No clinical signs of disease or distress were observed. The mice were placed in quarantine with daily inspections.

Day 4: The mice were individually examined and found to be free of any clinical signs of disease or distress. No deaths were recorded during the quarantine period. The mice were released to routine maintenance.

Day 8: 10% HP-β-CD was prepared by dissolving 5 g (2-hydroxypropyl)-β-cyclodextrin (Aldrich, Cat. 332593, lot MKBS9923V) was dissolved in 50 mL de-ionized water.

Day 11: Groups 1 and 2 were administered 10% HP-β-CD. Group 3: 7.59 mg dexamethasone 21-phosphate disodium salt (Sigma, Cat. D1159, lot 88H1020, SF 1.316) was dissolved in 5.767 mL HP-β-CD to prepare a 0.2 mg/mL solution. Group 4: 1 mg Example 7 was dissolved in 4.5 mL HP-β-CD and 7 µL 1 M HCI (MP Biomedicals, Cat. 194055, lot 3956H) with alternating sonication and vortexing. The pH was adjusted by addition of 5 µL 1 M NaOH and the volume brought up to 5 mL with 0.488 mL HP-β-CD to yield a 0.2 mg/mL solution. Group 5: 3 mg Example 7 was dissolved in 4.5 mL HP-β-CD and 20 µL 1 M HCI with alternating sonication and vortexing. The pH was adjusted by addition of 16 µL 1 M NaOH and the volume brought up to 5 mL with 0.464 mL HP-β-CD to yield a 0.6 mg/ mL solution. Group 6: 10 mg Example 7 was dissolved in 4.5 mL HP-β-CD and 66 µL 1 M HCI with alternating sonication and vortexing. The pH was adjusted by addition of 53 µL 1 M NaOH and the volume brought up to 5 mL with 0.381 mL HP-β-CD to yield a 2 mg/mL solution.

25.7 mg lipopolysaccharides (LPS) from E. coli strain 055:B5 (Sigma, Cat. L2880, lot 025M4040V) was dissolved in 2.57 mL sterile saline (Hospira, lot 56-836-FW) to prepare a 10 mg/mL solution.

Heparinized saline was prepared by addition of 3 mL heparin sodium (1000 U/mL, Sagent Pharmaceuticals, lot WG435N) to 297 mL sterile saline to prepare a 10 U/mL solution.

The mice were numbered, weighed, and dosed orally at 10 mL/kg as in Table 5.

TABLE 5

TREATMENT GROUPS

| Group | Treatment | Dose (mg/kg) |
|---|---|---|
| 1 | Vehicle | 10 mL/kg |
| 2 | Vehicle | 10 mL/kg |
| 3 | Dexamethasone | 10 mg/kg |
| 4 | Example 7 | 2 mg/kg |
| 5 | Example 7 | 6 mg/kg |
| 6 | Example 7 | 20 mg/kg |

An hour later, the mice were anesthetized, a midline incision was made in the neck, the muscle layers separated by blunt dissection, and 2 mL/kg LPS (20 mg/kg) injected into the trachea (Groups 2-6). The incision was closed with wound clips and the mice returned to cages. Group 1 received 2 mL/kg saline.

Six hours after LPS/saline injection, the mice were anesthetized, the wound clips removed, the trachea was cannulated with a 23 G needle blunt, and the lungs lavaged eight times with 0.5 mL heparinized saline. The lavage was pooled, gently inverted, and a sample retained for white blood cell (WBC) counts and differential analysis. The remainder of the lavage was centrifuged, the supernatants dispensed to one aliquot in labelled Eppendorf tubes which were stored at -80° C. The carcasses were disposed of appropriately.

Day 14: The aliquot of lung lavage supernatant was equilibrated to room temperature, diluted 1:2 and assayed for TNFα by ELISA (R&D Systems, Cat. MTA00B, lot 332141).

TABLE 6

TNFA STANDARD CURVE

| TNFα (pg/ mL) | Absorbance |
|---|---|
| 0 | 0.027 |
| 10.9 | 0.074 |
| 21.9 | 0.119 |
| 43.8 | 0.19 |
| 87.5 | 0.401 |
| 175 | 0.731 |
| 350 | 1.34 |
| 700 | 2.545 |

TABLE 7

EFFECT OF ORAL PRETREATMENT WITH EXAMPLE 7 ON LPS-INDUCED PULMONARY NEUTROPHIL INFILTRATION

| Group | Statistic | WBC/ mL | Neutrophils |
|---|---|---|---|
| 1 | Mean SD p-value | 5.4E+05 2.7E+05 *** | 16 15 *** |
| 2 | Mean SD | 9.6E+06 1.6E+06 | 794 442 |
| 3 | Mean SD p-value | 1.2E+06 1.7E+06 *** | 55 67 *** |
| 4 | Mean SD p-value | 9.2E+06 2.1E+06 ns | 583 204 Ns |
| 5 | Mean SD p-value | 2.9E+06 2.0E+06 ** | 26 65 *** |
| 6 | Mean SD p-value | 6.7E+05 2.8E+05 *** | 24 29 *** |

Significance (p-value) was calculated vs Group 2 by one-way ANOVA followed by Dunnett’s post hoc analysis. ns = non-significant ***p<0.001

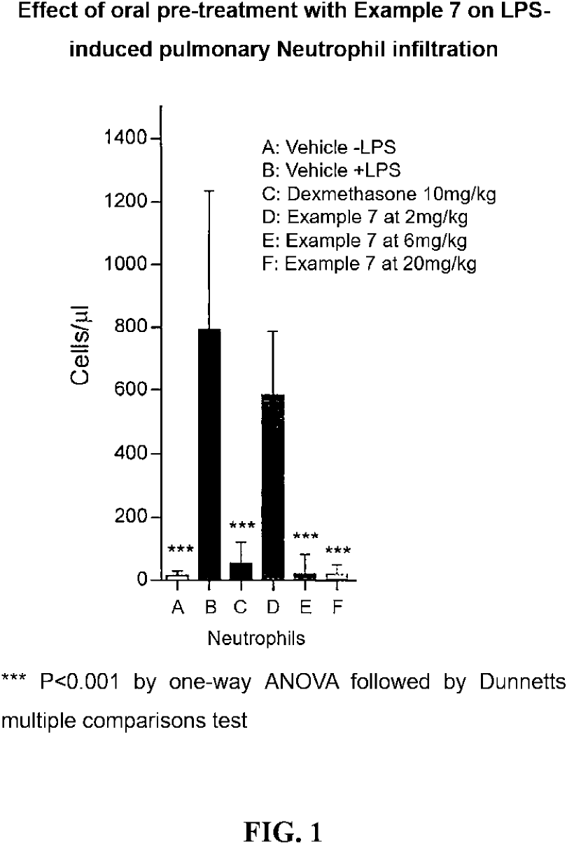

The results can be seen in FIG. 1.

TABLE 8

EFFECT OF TREATMENT ON AVERAGE LAVAGE TNFα (PG/ML)

| Group | Statistic | TNFα |
|---|---|---|
| 1 | Mean SD p-value | 0.03 0.03 *** |
| 2 | Mean SD | 375.06 29.83 |
| 3 | Mean SD p-value | 7.39 1.69 *** |
| 4 | Mean SD p-value | 337.00 30.33 ns |
| 5 | Mean SD p-value | 132.90 17.18 *** |
| 6 | Mean SD p-value | 98.87 11.91 *** |

Significance (p-value) was calculated vs Group 2 by One-way ANOVA followed by Dunnett’s post hoc analysis. ns = non-significant ***p<0.001

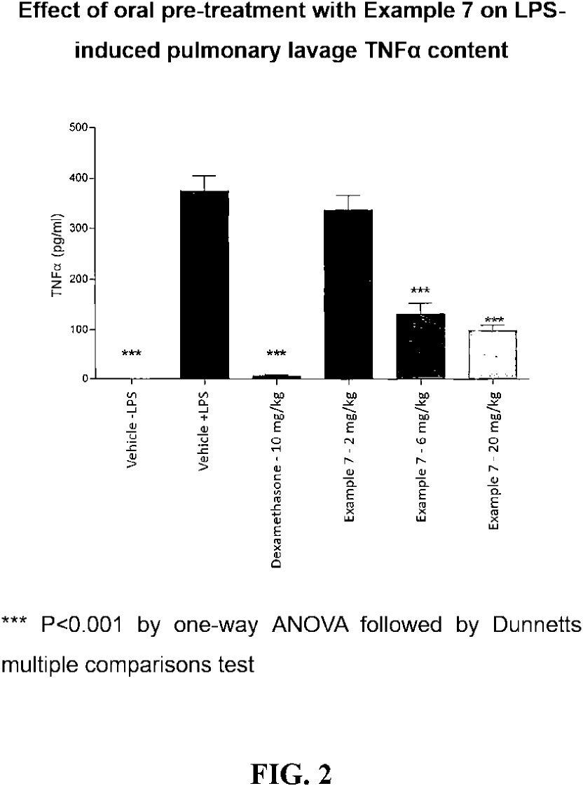

The results can be seen in FIG. 2.

### F. LPS-Induced Pulmonary Inflammation

In response to endotracheal administration of 20 mg/mL LPS, the percentage of neutrophils in the lavage fluid was 10-fold higher in the LPS-treated mice (Group 2), than that observed in the saline-treated mice (Group 1). The lavage fluid in the LPS-challenge mice contained ~375 pg/mL TNFα.

### G. Effect of Prophylactic Oral Dexamethasone Administration (Group 3)

One hour pre-treatment with orally administered 10 mg/kg dexamethasone prior to LPS challenge (Group 3) resulted in a significant 87% reduction in inflammatory cell infiltration which corresponded to a 98% reduction in lavage TNFα content, relative to the LPS control mice (Group 2). The percentage of neutrophils in the lavage fluid was reduced to values similar to those observed in the saline-treated mice (Group 1).

### H. Effect of Prophylactic Oral Example 7 Administration (Groups 4-6)

One hour pre-treatment with orally administered Example 7 prior to LPS challenge resulted in a significant dose-dependent reduction in all measured parameters. At the highest dose (20 mg/kg, Group 6), a significant reduction in infiltrating inflammatory cells (93%) and lavage TNFα content (74%) was recorded. At the intermediate dose (6 mg/kg, Group 5), a significant reduction in infiltrating inflammatory cells (70%) and lavage TNFα content (65%) was recorded. The percentage of neutrophils in the lavage fluid was reduced to values similar to those observed in the saline-treated mice (Group 1; 93%) with both the 6 mg/kg (97%) and 20 mg/kg (97%) dose treatment regimen. The lowest dose (2 mg/kg, Group 4) showed a slight reduction in infiltrating inflammatory cells (3%), TNFα content (10%) and neutrophils (27%) which did not reach statistical significance.

The results show that administration of a compound of Formula (I) provides a significant dose-dependent reduction of pulmonary inflammatory cells and lung lavage TNFα content in response to endotracheal LPS challenge. The methods of the invention are effective to treat pulmonary inflammation, and in particular inflammation (and hyperinflammation) associated with a coronavirus infection.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating or preventing pulmonary inflammation in a subject in need thereof, the method comprising administering to the subject a compound having a structure represented by a formula: 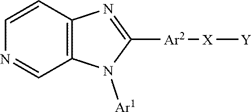
wherein Ar¹ is selected from the group consisting of phenyl and heteroaryl, each of which is optionally substituted with one or more halogen groups;
wherein Ar² is selected from the group consisting of phenyl and heteroaryl, each of which is optionally substituted with one or more halogen groups;
wherein X is selected from the group consisting of 3- to 7-membered heterocyclic ring and 3 to 7-membered cycloalkyl ring, each of which is optionally substituted with one or more halogen or C1-C4 alkyl groups;
wherein Y is selected from the group consisting of C1-C4 alkyl, C1-C4 haloalkyl, —CN, —OR¹, —C(O)R¹, —NR²R³, —NR¹C(O)R⁴, —C(O)NR²R³, —S(O)R¹, —SO₂R¹, —S(O)NR²R³, and —SO₂NR²R³;
wherein R¹ is selected from the group consisting of H, C1-C4 alkyl, and C1-C4 haloalkyl;
wherein each of R² and R³ are independently selected from the group consisting of H, C1-C4 alkyl, and C1-C4 haloalkyl,
or wherein each of R² and R³ together with the nitrogen to which they are attached comprise a 3- to 7-membered heterocyclic ring optionally substituted with one or more halogen groups; and
wherein R⁴ is selected from the group consisting of H, C1-C4 alkyl, and C1-C4 haloalkyl,
or a pharmaceutically acceptable salt thereof.

2. The method of claim 1, wherein Ar¹ is phenyl monosubstituted with a halogen groups.

3. (canceled)

4. (canceled)

5. The method of claim 1, wherein Ar¹ is phenyl monosubstituted with a fluoro group.

6. The method of claim 1, wherein Ar² is selected from pyridinyl and pyrimidinyl, and is substituted with 0, 1, 2, or 3 halogen groups.

7. The method of claim 1, wherein Ar² is unsubstituted pyridinyl.

8. The method of claim 1, wherein X is a 3 to 7-membered heterocyclic ring.

9. The method of claim 1, wherein X is a 6-membered heterocyclic ring having a structure: 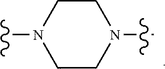
.

10. The method of claim 1, wherein Y is —SO₂R¹.

11. The method of claim 1, wherein the compound has a structure represented by a formula: 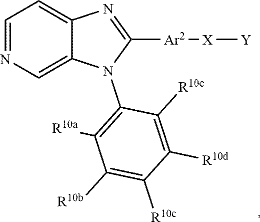
wherein each of R^{10a}, R^{10b}, R^{10c}, R^{10d}, and R^{10e} is independently selected from hydrogen and halogen, provided that at least two of R^{10a}, R^{10b}, R^{10c}, R^{10d}, and R^{10e} are hydrogen.

12. The method of claim 1, wherein the compound has a structure represented by a formula: 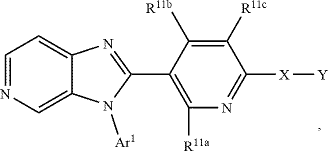
wherein each of R^{11a}, R^{11b}, and R^{11c} is independently selected from hydrogen and halogen.

13. The method of claim 1, wherein the compound has a structure represented by a formula: 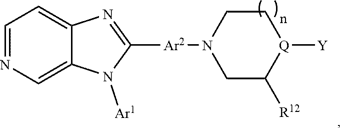
wherein n is 1 or 2;
wherein Q is O, N, or CH; and
wherein R¹² is H, halogen, or C1-C4 alkyl.

14. The method of claim 1, wherein the compound is selected from: 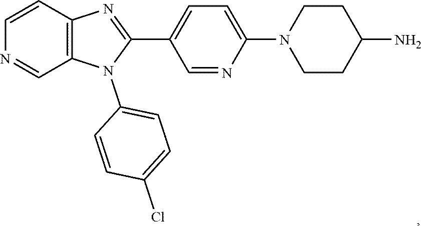 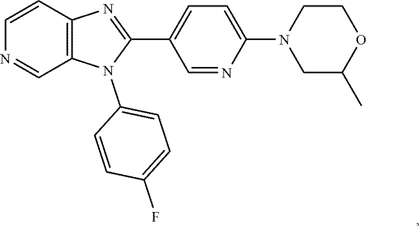 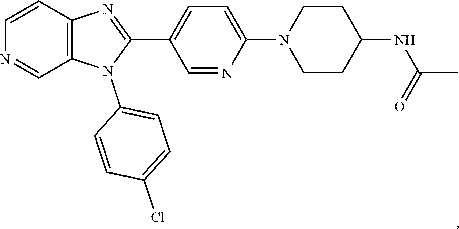 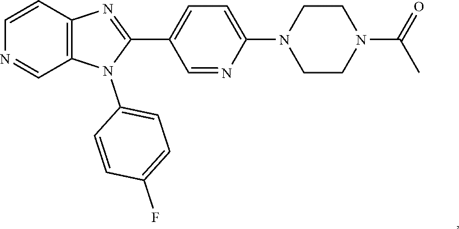 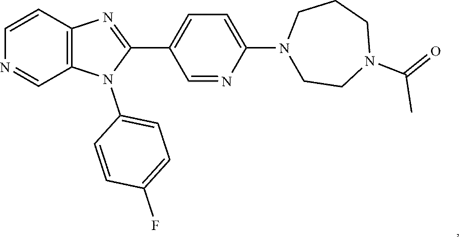 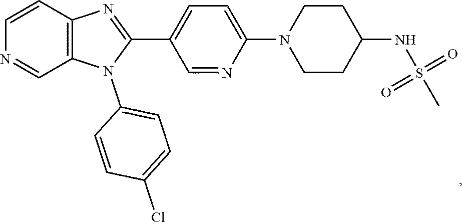 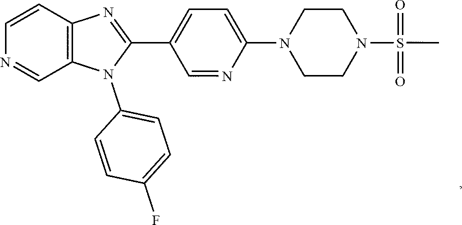 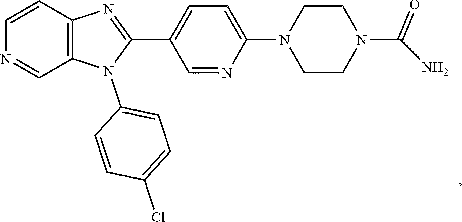 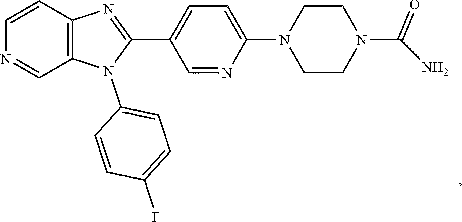 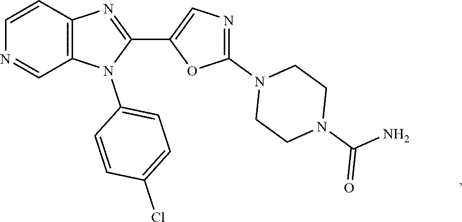 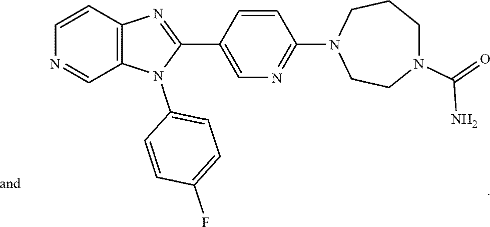
.

15. The method of claim 1, wherein the compound is: 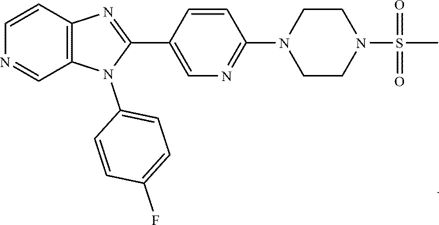
.

16. The method of claim 1, wherein the subject has been previously diagnosed as having acute lung injury (ALI) and/or acute respiratory distress syndrome (ARDS).

17. The method of claim 1, wherein the pulmonary inflammation is caused by a cytokine surge.

18. The method of claim 1, wherein the pulmonary inflammation is caused by a coronavirus.

19. The method of claim 16, wherein the coronavirus is severe acute respiratory syndrome coronavirus (SARS-CoV), severe acute respiratory syndrome coronavirus 2 (SARS-CoV-2), or Middle East respiratory syndrome coronavirus (MERS-CoV).

20. The method of claim 16, wherein the coronavirus is SARS-CoV-2.
